(19) United States
(12) Patent Application Publication
(10) Pub. No.: US 2019/0348152 A1
(43) Pub. Date: Nov. 14, 2019

(54) MACHINE LEARNING ENABLED BIOLOGICAL POLYMER ASSEMBLY

(71) Applicant: Quantum-Si Incorporated, Guilford, CT (US)

(72) Inventor: Minh Duc Cao, Guilford, CT (US)

(73) Assignee: Quantum-Si Incorporated, Guilford, CT (US)

(21) Appl. No.: 16/411,056

(22) Filed: May 13, 2019

Related U.S. Application Data
(60) Provisional application No. 62/671,884, filed on May 15, 2018.
(60) Provisional application No. 62/671,260, filed on May 14, 2018.

(51) Int. Cl. G16B 40/00; G16B 15/00; G16B 30/00; G16B 50/00

(52) U.S. Cl. CPC G16B 40/00; G16B 50/00; G16B 30/00; G16B 15/00

(57) ABSTRACT

Described herein are machine learning techniques for generating biological polymer assemblies of macromolecules. For example, the system may use machine learning techniques to generate a genome assembly of an organism's DNA, a gene sequence of a portion of an organism's DNA, or an amino acid sequence of a protein. The system may access biological polymer sequences generated by a sequencing device and an assembly generated from the sequences. The system may generate input to a machine learning model using the sequences and the assembly. The system may provide the input to the machine learning model to obtain a corresponding output. The system may use the corresponding output to identify biological polymers at locations in the assembly, and then update the assembly to indicate the identified biological polymers at the locations in the assembly to obtain an updated assembly.

### RELATED APPLICATIONS

This Application claims the benefit under 35 U.S.C. § 119(e) to U.S. Provisional Application Ser. No. 62/671,884, entitled “DEEP LEARNING MODEL TO IMPROVE SPEED AND ACCURACY OF GENOME ASSEMBLY”, filed May 15, 2018, and to U.S. Provisional Application Ser. No. 62/671,260, entitled “DEEP LEARNING MODEL TO IMPROVE SPEED AND ACCURACY OF GENOME ASSEMBLY”, filed May 14, 2018, each of which is incorporated herein by reference in its entirety.

### BACKGROUND

The present disclosure relates to generating an assembly of biological polymers (e.g., a genome assembly, nucleotide sequence, or protein sequence) of a macromolecule (e.g., a nucleic acid or protein). Sequencing devices may generate sequencing data that can be used to generate an assembly. As an example, the sequencing data may include nucleotide sequences of DNA from a biological sample which can be used to assemble a genome (in whole or in part). As another example, the sequencing data may include amino acid sequences which can be used to assemble a protein sequence (in whole or in part).

### SUMMARY

According to one aspect, a method of generating a biological polymer assembly of a macromolecule is provided. The method comprises: using at least one computer hardware processor to perform; accessing a plurality of biological polymer sequences and an assembly indicating biological polymers present at respective assembly locations; generating, using the plurality of biological polymer sequences and the assembly, a first input to be provided to a trained deep learning model; providing the first input to the trained deep learning model to obtain a corresponding first output indicating, for each of a first plurality of assembly locations, one or more likelihoods that each of one or more respective biological polymers is present at the location; identifying biological polymers at the first plurality of assembly locations using the first output of the trained deep learning model; and updating the assembly to indicate the identified biological polymers at the first plurality of assembly locations to obtain an updated assembly.

According to one embodiment, the macromolecule comprises a protein, the plurality of biological polymer sequences comprises a plurality of amino acid sequences, and the assembly indicates amino acids at respective assembly locations.

According to one embodiment, the macromolecule comprises a nucleic acid, the plurality of biological polymer sequences comprises a plurality of nucleotide sequences, and the assembly indicates nucleotides at respective assembly locations.

According to one embodiment, the assembly indicates a first nucleotide at a first one of the first plurality of assembly locations; identifying the biological polymers at the first plurality of assembly locations comprises identifying a second nucleotide at the first assembly location; and updating the assembly comprises updating the assembly to indicate the second nucleotide at the first assembly location.

According to one embodiment, the method further comprises, after updating the assembly to obtain the updated assembly: aligning the plurality of nucleotide sequences to the updated assembly; generating, using the plurality of nucleotide sequences and the updated assembly, a second input to be provided to the trained deep learning model; providing the second input to the trained deep learning model to obtain a corresponding second output indicating, for each of a second plurality of assembly locations, one or more likelihoods that each of one or more respective nucleotides is present at the location; identifying nucleotides at the second plurality of assembly locations based on the second output of the trained deep learning model; and updating the updated assembly to indicate the identified nucleotides at the second plurality of assembly locations to obtain a second updated assembly.

According to one embodiment, the method further comprises aligning the plurality of nucleotide sequences to the assembly. According to one embodiment, the plurality of nucleotide sequences comprises at least 5 nucleotide sequences. According to one embodiment, the plurality of nucleotide sequences comprises at least 9 nucleotide sequences. According to one embodiment, the plurality of nucleotide sequences comprises at least 10 nucleotide sequences.

According to one embodiment, generating the first input to the trained deep learning model comprises: selecting the first plurality of assembly locations; and generating the first input based on the selected first plurality of assembly locations. According to one embodiment, selecting the first plurality locations in the assembly comprises: determining likelihoods that the assembly incorrectly indicates nucleotides at the first plurality of assembly locations; and selecting the first plurality of assembly locations using the determined likelihoods.

According to one embodiment, generating the first input to be provided to the trained deep learning model comprises comparing respective ones of the plurality of nucleotide sequences to the assembly. According to one embodiment, generating the first input to be provided to the trained deep learning model to identify a nucleotide at a first one of the first plurality of assembly locations comprises: for each of multiple nucleotides at each of one or more assembly locations in a neighborhood of the first assembly location: determining a count indicating a number of the plurality of nucleotide sequences that indicate that the nucleotide is at the location; determining a reference value based on whether the assembly indicates the nucleotide at the location; determining an error value indicating a difference between the count and the reference value; and including the reference value and the error value in the first input.

According to one embodiment, determining the reference value based on whether the assembly indicates the nucleotide at the location comprises: determining the reference value to be a first value when the assembly indicates the nucleotide at the location; and determining the reference value to be a second value when the assembly does not indicate the nucleotide at the location. According to one embodiment, the first value is a number of the plurality of nucleotide sequences; and the second value is 0.

According to one embodiment, generating the first input to be provided to the trained deep learning model comprises arranging values into a data structure having columns, wherein: a first column holds reference values and error values determined for the multiple nucleotides at the first assembly location; and a second column holds reference values and error values determined for the multiple nucleotides at a second one of the one or more assembly locations in the neighborhood of the first assembly location. According to one embodiment, the one or more assembly locations in the neighborhood of the first assembly location comprise at least two assembly locations separate from the first assembly location.

According to one embodiment, the one or more likelihoods that each of the one or more respective biological polymers is present at the assembly location comprises, for each of multiple nucleotides, a likelihood that the nucleotide is present at the assembly location; and identifying biological polymers at the first plurality of assembly locations comprises identifying a nucleotide at a first one of the first plurality of assembly locations to be a first one of the multiple nucleotides by determining that a likelihood that the first nucleotide is present at the first location is greater than a likelihood that a second one of the multiple nucleotides is present at the first assembly location.

According to one embodiment, the method further comprises generating the assembly from the plurality of nucleotide sequences. According to one embodiment, generating the assembly from the plurality of nucleotide sequences comprises determining a consensus sequence from the plurality of nucleotide sequences to be the assembly. According to one embodiment, generating the assembly from the plurality of nucleotide sequences comprises applying an overlap layout consensus (OLC) algorithm to the plurality of nucleotide sequences.

According to one embodiment, the method further comprises: accessing training data including biological polymer sequences obtained from sequencing a reference macromolecule and a predetermined assembly of the reference macromolecule; and training a deep learning model using the training data to obtain the trained deep learning model. According to one embodiment, the reference macromolecule is different from the macromolecule. According to one embodiment, the deep learning model comprises a convolutional neural network (CNN).

According to another aspect, a system for generating a biological polymer assembly of a macromolecule is provided. The system comprises: at least one computer hardware processor; and at least one non-transitory computer-readable storage medium storing instructions that, when executed by the at least one computer hardware processor, cause the at least one computer hardware processor to perform: accessing a plurality of biological polymer sequences and an assembly indicating biological polymers present at respective assembly locations; generating, using the plurality of biological polymer sequences and the assembly, a first input to be provided to a trained deep learning model; providing the first input to the trained deep learning model to obtain a corresponding first output indicating, for each of a first plurality of assembly locations, one or more likelihoods that each of one or more respective biological polymers is present at the location; identifying biological polymers at the first plurality of assembly locations using the first output of the trained deep learning model; and updating the assembly to indicate the identified biological polymers at the first plurality of assembly locations to obtain an updated assembly.

According to one embodiment, the macromolecule comprises a protein, the plurality of biological polymer sequences comprises a plurality of amino acid sequences, and the assembly indicates amino acids at respective assembly locations.

According to one embodiment, the macromolecule comprises a nucleic acid, the plurality of biological polymer sequences comprises a plurality of nucleotide sequences, and the assembly indicates nucleotides at respective assembly locations.

According to one embodiment, the assembly indicates a first nucleotide at a first one of the first plurality of assembly locations; identifying the biological polymers at the first plurality of assembly locations comprises identifying a second nucleotide at the first assembly location; and updating the assembly comprises updating the assembly to indicate the second nucleotide at the first assembly location.

According to one embodiment, the instructions further cause the at least one computer hardware processor to perform, after updating the assembly to obtain the updated assembly: aligning the plurality of nucleotide sequences to the updated assembly; generating, using the plurality of nucleotide sequences and the updated assembly, a second input to be provided to the trained deep learning model; providing the second input to the trained deep learning model to obtain a corresponding second output indicating, for each of a second plurality of assembly locations, one or more likelihoods that each of one or more respective nucleotides is present at the location; identifying nucleotides at the second plurality of assembly locations based on the second output of the trained deep learning model; and updating the updated assembly to indicate the identified nucleotides at the second plurality of assembly locations to obtain a second updated assembly.

According to one embodiment, the instructions further cause the at least one computer hardware processor to perform aligning the plurality of nucleotide sequences to the assembly. According to one embodiment, the plurality of nucleotide sequences comprises at least 5 nucleotide sequences. According to one embodiment, the plurality of nucleotide sequences comprises at least 9 nucleotide sequences. According to one embodiment, the plurality of nucleotide sequences comprises at least 10 nucleotide sequences.

According to one embodiment, generating the first input to the trained deep learning model comprises: selecting the first plurality of assembly locations; and generating the first input based on the selected first plurality of assembly locations. According to one embodiment, selecting the first plurality locations in the assembly comprises: determining likelihoods that the assembly incorrectly indicates nucleotides at the first plurality of assembly locations; and selecting the first plurality of assembly locations using the determined likelihoods.

According to one embodiment, generating the first input to be provided to the trained deep learning model comprises comparing respective ones of the plurality of nucleotide sequences to the assembly. According to one embodiment, generating the first input to be provided to the trained deep learning model to identify a nucleotide at a first one of the first plurality of assembly locations comprises: for each of multiple nucleotides at each of one or more assembly locations in a neighborhood of the first assembly location: determining a count indicating a number of the plurality of nucleotide sequences that indicate that the nucleotide is at the location; determining a reference value based on whether the assembly indicates the nucleotide at the location; determining an error value indicating a difference between the count and the reference value; and including the reference value and the error value in the first input. According to one embodiment, determining the reference value based on whether the assembly indicates the nucleotide at the location comprises: determining the reference value to be a first value when the assembly indicates the nucleotide at the location; and determining the reference value to be a second value when the assembly does not indicate the nucleotide at the location. According to one embodiment, the first value is a number of the plurality of nucleotide sequences; and the second value is 0. According to one embodiment, generating the first input to be provided to the trained deep learning model comprises arranging values into a data structure having columns, wherein: a first column holds reference values and error values determined for the multiple nucleotides at the first assembly location; and a second column holds reference values and error values determined for the multiple nucleotides at a second one of the one or more assembly locations in the neighborhood of the first assembly location. According to one embodiment, the one or more assembly locations in the neighborhood of the first assembly location comprise at least two assembly locations separate from the first assembly location.

According to one embodiment, the one or more likelihoods that each of the one or more respective biological polymers is present at the assembly location comprises, for each of multiple nucleotides, a likelihood that the nucleotide is present at the assembly location; and identifying biological polymers at the first plurality of assembly locations comprises identifying a nucleotide at a first one of the first plurality of assembly locations to be a first one of the multiple nucleotides by determining that a likelihood that the first nucleotide is present at the first location is greater than a likelihood that a second one of the multiple nucleotides is present at the first assembly location.

According to one embodiment, the instructions further cause the at least one computer hardware processor to perform generating the assembly from the plurality of nucleotide sequences. According to one embodiment, generating the assembly from the plurality of nucleotide sequences comprises determining a consensus sequence from the plurality of nucleotide sequences to be the assembly. According to one embodiment, generating the assembly from the plurality of nucleotide sequences comprises applying an overlap layout consensus (OLC) algorithm to the plurality of nucleotide sequences.

According to one embodiment, the instructions further cause the at least one computer hardware processor to perform: accessing training data including biological polymer sequences obtained from sequencing a reference macromolecule and a predetermined assembly of the reference macromolecule; and training a deep learning model using the training data to obtain the trained deep learning model. According to one embodiment, the reference macromolecule is different from the macromolecule. According to one embodiment, the deep learning model comprises a convolutional neural network (CNN).

According to another aspect, a non-transitory computer-readable storage medium is provided. The non-transitory computer-readable storage medium stores instructions that, when executed by at least one computer hardware processor, cause the at least one computer hardware processor to perform a method of generating a biological polymer assembly of a macromolecule. The method comprises: accessing a plurality of biological polymer sequences and an assembly indicating biological polymers present at respective assembly locations; generating, using the plurality of biological polymer sequences and the assembly, a first input to be provided to a trained deep learning model; providing the first input to the trained deep learning model to obtain a corresponding first output indicating, for each of a first plurality of assembly locations, one or more likelihoods that each of one or more respective biological polymers is present at the location; identifying biological polymers at the first plurality of assembly locations using the first output of the trained deep learning model; and updating the assembly to indicate the identified biological polymers at the first plurality of assembly locations to obtain an updated assembly.

According to one embodiment, the macromolecule comprises a protein, the plurality of biological polymer sequences comprises a plurality of amino acid sequences, and the assembly indicates amino acids at respective assembly locations.

According to one embodiment, the macromolecule comprises a nucleic acid, the plurality of biological polymer sequences comprises a plurality of nucleotide sequences, and the assembly indicates nucleotides at respective assembly locations.

According to one embodiment, the assembly indicates a first nucleotide at a first one of the first plurality of assembly locations; identifying the biological polymers at the first plurality of assembly locations comprises identifying a second nucleotide at the first assembly location; and updating the assembly comprises updating the assembly to indicate the second nucleotide at the first assembly location.

According to one embodiment, the method further comprises, after updating the assembly to obtain the updated assembly: aligning the plurality of nucleotide sequences to the updated assembly; generating, using the plurality of nucleotide sequences and the updated assembly, a second input to be provided to the trained deep learning model; providing the second input to the trained deep learning model to obtain a corresponding second output indicating, for each of a second plurality of assembly locations, one or more likelihoods that each of one or more respective nucleotides is present at the location; identifying nucleotides at the second plurality of assembly locations based on the second output of the trained deep learning model; and updating the updated assembly to indicate the identified nucleotides at the second plurality of assembly locations to obtain a second updated assembly.

According to one embodiment, the method further comprises aligning the plurality of nucleotide sequences to the assembly. According to one embodiment, the plurality of nucleotide sequences comprises at least 5 nucleotide sequences. According to one embodiment, the plurality of nucleotide sequences comprises at least 9 nucleotide sequences. According to one embodiment, the plurality of nucleotide sequences comprises at least 10 nucleotide sequences.

According to one embodiment, generating the first input to the trained deep learning model comprises: selecting the first plurality of assembly locations; and generating the first input based on the selected first plurality of assembly locations. According to one embodiment, selecting the first plurality locations in the assembly comprises: determining likelihoods that the assembly incorrectly indicates nucleotides at the first plurality of assembly locations; and selecting the first plurality of assembly locations using the determined likelihoods.

According to one embodiment, generating the first input to be provided to the trained deep learning model comprises comparing respective ones of the plurality of nucleotide sequences to the assembly. According to one embodiment, generating the first input to be provided to the trained deep learning model to identify a nucleotide at a first one of the first plurality of assembly locations comprises: for each of multiple nucleotides at each of one or more assembly locations in a neighborhood of the first assembly location: determining a count indicating a number of the plurality of nucleotide sequences that indicate that the nucleotide is at the location; determining a reference value based on whether the assembly indicates the nucleotide at the location; determining an error value indicating a difference between the count and the reference value; and including the reference value and the error value in the first input. According to one embodiment, determining the reference value based on whether the assembly indicates the nucleotide at the location comprises: determining the reference value to be a first value when the assembly indicates the nucleotide at the location; and determining the reference value to be a second value when the assembly does not indicate the nucleotide at the location. According to one embodiment, the first value is a number of the plurality of nucleotide sequences; and the second value is 0. According to one embodiment, generating the first input to be provided to the trained deep learning model comprises arranging values into a data structure having columns, wherein: a first column holds reference values and error values determined for the multiple nucleotides at the first assembly location; and a second column holds reference values and error values determined for the multiple nucleotides at a second one of the one or more assembly locations in the neighborhood of the first assembly location. According to one embodiment, the one or more assembly locations in the neighborhood of the first assembly location comprise at least two assembly locations separate from the first assembly location.

According to one embodiment, the one or more likelihoods that each of the one or more respective biological polymers is present at the assembly location comprises, for each of multiple nucleotides, a likelihood that the nucleotide is present at the assembly location; and identifying biological polymers at the first plurality of assembly locations comprises identifying a nucleotide at a first one of the first plurality of assembly locations to be a first one of the multiple nucleotides by determining that a likelihood that the first nucleotide is present at the first location is greater than a likelihood that a second one of the multiple nucleotides is present at the first assembly location.

According to one embodiment, the method further comprises generating the assembly from the plurality of nucleotide sequences. According to one embodiment, generating the assembly from the plurality of nucleotide sequences comprises determining a consensus sequence from the plurality of nucleotide sequences to be the assembly. According to one embodiment, generating the assembly from the plurality of nucleotide sequences comprises applying an overlap layout consensus (OLC) algorithm to the plurality of nucleotide sequences.

According to one embodiment, the method further comprises: accessing training data including biological polymer sequences obtained from sequencing a reference macromolecule and a predetermined assembly of the reference macromolecule; and training a deep learning model using the training data to obtain the trained deep learning model. According to one embodiment, the reference macromolecule is different from the macromolecule. According to one embodiment, the deep learning model comprises a convolutional neural network (CNN).

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIGS. 1A-C show systems in which aspects of the technology described herein may be implemented, in accordance with some embodiments of the technology described herein.

FIGS. 2A-D show embodiments of an assembly system, in accordance with some embodiments of the technology described herein.

FIG. 3A is an example process 300 for training a machine learning model for generating a biological polymer assembly, in accordance with some embodiments of the technology described herein.

FIG. 3B is an example process 310 for using the machine learning model obtained by the process of FIG. 3A to generate a biological polymer assembly, in accordance with some embodiments of the technology described herein.

FIGS. 4A-C illustrate an example of generating input to a machine learning model, in accordance with some embodiments of the technology described herein.

FIG. 5 illustrates an example of updating a biological polymer assembly, in accordance with some embodiments of the technology described herein.

FIG. 6 illustrates the structure of an illustrative convolutional neural network (CNN) model used for generating a biological polymer assembly, in accordance with some embodiments of the technology described herein.

FIG. 7 shows performance of assembly techniques, implemented in accordance with some embodiments of the technology described herein, relative to conventional techniques.

FIG. 8 is a block diagram of an illustrative computing device 800 that may be used in implementing some embodiments of the technology described herein.

### DETAILED DESCRIPTION

A macromolecule may be a protein or protein fragment, a DNA molecule (of any type of DNA) or fragment, or an RNA molecule (of any type of RNA) or fragment. A biological polymer may be an amino acid (e.g., when a macromolecule is a protein or a fragment thereof), or a nucleotide (e.g., when a macromolecule is DNA, RNA, or a fragment thereof).

The inventors have developed a system that uses machine learning techniques to generate biological polymer assemblies of macromolecules. For example, the system developed by the inventors may be configured to employ machine learning techniques to generate a genome assembly of an organism's DNA. As another example, the system developed by the inventors may be configured to employ machine learning techniques to generate an amino acid sequence of a protein.

In some embodiments, the system may access one or more biological polymer sequences (e.g., generated by a sequencing device) and an initial assembly generated from the sequences. The assembly may indicate presence of biological polymers (e.g., nucleotides, amino acids) at respective assembly locations. The system may correct errors in biological polymer indications of the initial assembly by: (1) generating input to be provided to a machine learning model using the sequences and the initial assembly; (2) providing the input to a trained machine learning model to obtain a corresponding output; and (3) updating the initial assembly using the output obtained from the machine learning model to obtain an updated assembly. The updated assembly may have fewer errors in biological polymer indications than the initial assembly.

In some embodiments, an assembly may comprise multiple locations and indications of biological polymers (e.g., nucleotides or amino acids) at respective locations. As an example, an assembly may be a genome assembly indicating nucleotides at locations in an organism's genome. As another example, an assembly may be a gene sequence indicating a sequence of nucleotides of a portion of an organism's DNA. As another example, an assembly may be an amino acid sequence of a protein (also referred to as a “protein sequence”). A biological polymer may be a nucleotide, amino acid, or any other type of biological polymer. A biological polymer sequence may also be referred to herein as a “sequence” or a “read.”

Some conventional biological polymer assembly techniques may utilize sequencing technology to generate biological polymer sequences of a macromolecule (e.g., DNA, RNA, or a protein), and generate an assembly of the macromolecule using the generated sequences. For example, a sequencing device may generate nucleotide sequences from a DNA samples of an organism, which sequences may in turn be used to generate a genome assembly of the organism's DNA. As another example, a sequencing device may generate amino acid sequences of a protein sample, which sequences may in turn be used to assemble a longer amino acid sequence for the protein. A computing device may apply an assembly algorithm to sequences generated by a sequencing device to generate the assembly. For example, the computing device may apply the overlap layout consensus (OLC) assembly algorithm to nucleotide sequences of a DNA sample to generate an organism's genome assembly or portion thereof.

One type of sequencing technology used for generating nucleotide sequences from a nucleic acid sample is second generation sequencing (also known as “short-read sequencing”) which generates nucleotide sequences of less than 1000 nucleotides (i.e., “short reads”). Sequencing technology has now advanced to third generation sequencing (also known as “long-read sequencing”) which generates nucleotide sequences of 1000 or more nucleotides (i.e., “long reads”), and provides larger portions of an assembly than does second generation sequencing. However, the inventors have recognized that third generation sequencing is less accurate than second generation sequencing and, as a result, assemblies generated from long reads are less accurate than those generated from short reads. The inventors have also recognized that conventional error correction techniques to improve assembly accuracy are computationally expensive and time consuming. Accordingly, the inventors have developed machine learning techniques for correcting errors in assemblies that: (1) improve the accuracy of assemblies generated from third generation sequencing; and (2) are more efficient than conventional error correction techniques.

Some embodiments described herein address all of the above-described issues that the inventors have recognized with generation of assemblies. However, it should be appreciated that not every embodiment described herein addresses every one of these issues. It should also be appreciated that embodiments of the technology described herein may be used for purposes other than addressing the above-discussed issues of biological polymer assembly. As one example, embodiments of the technology described herein may be used to improve accuracy of protein sequences generated from amino acid sequences. As another example, embodiments of the technology described herein may be used to improve accuracy of assemblies generated from short reads.

In some embodiments, the system may be configured to: (1) access an assembly (e.g., generated from a plurality of biological polymer sequences) indicating biological polymers present at respective assembly locations; (2) generate, using the plurality of biological polymer sequences and the assembly, a first input to be provided to a trained deep learning model; (3) provide the first input to the trained deep learning model to obtain a corresponding first output indicating, for each of a first plurality of assembly locations, one or more likelihoods (e.g., probabilities) that each of one or more respective biological polymers is present at the assembly location; (4) identify biological polymers at the first plurality of assembly locations using the first output of the trained deep learning model; and (5) update the assembly to indicate the identified biological polymers at the first plurality of assembly locations to obtain an updated assembly. In some embodiments, the system may be configured to align the plurality of biological polymer sequences to the assembly.

In some embodiments, the macromolecule may be a protein, the plurality of biological polymer sequence may be a plurality of amino acid sequences, and the assembly indicates amino acids at respective assembly locations. In some embodiments, the macromolecule may be a nucleic acid (e.g., DNA, RNA), the plurality of biological sequences may be nucleotide sequences, and the assembly indicates nucleotides at respective assembly locations.

In some embodiments, the assembly indicates a first nucleotide (e.g., adenine) at a first one of the plurality of assembly locations. Identifying biological polymers at the first plurality of assembly locations comprises identifying a second nucleotide (e.g., thymine) at the first assembly location that is different from the first nucleotide; and updating the assembly comprises updating the assembly to indicate the second nucleotide (e.g., thymine) at the first assembly location.

In some embodiments, the system may be configured to perform multiple iterations of updates. The system may be configured to, after updating the assembly to obtain the updated assembly: (1) align the plurality of nucleotide sequences to the updated assembly; (2) generate, using the plurality of nucleotide sequences and the updated assembly, a second input to be provided to the trained deep learning model; (3) provide the second input to the trained deep learning model to obtain a corresponding second output indicating, for each of a second plurality of assembly locations, one or more likelihoods (e.g., probabilities) that each of one or more respective nucleotides is present at the assembly location; (4) identify nucleotides at the second plurality of assembly locations based on the second output of the trained deep learning model; and (5) update the updated assembly to indicate the identified nucleotides at the second plurality of assembly locations to obtain a second updated assembly.

In some embodiments, the system may be configured to generate the first input to the trained deep learning model by: (1) selecting the first plurality of assembly locations; and (2) generating the first input based on the selected first plurality of assembly locations. In some embodiments, the system may be configured to select the first plurality of assembly locations by: (1) determining likelihoods that the assembly incorrectly indicates nucleotides at the first plurality of assembly locations; and (2) selecting the first plurality of assembly locations using the determined likelihoods.

In some embodiments, the system may be configured to generate the first input to be provided to the trained deep learning model by comparing respective ones of the plurality of nucleotide sequences to the assembly (e.g., to determine values of one or more features). In some embodiments, the system may be configured to generate the first input to identify a nucleotide at a first one of the first plurality of assembly locations by, for each of multiple nucleotides at each of one or more assembly locations in a neighborhood of the first assembly location: (1) determining a count indicating a number of the plurality of nucleotide sequences that indicate that the nucleotide is at the assembly location; (2) determining a reference value based on whether the assembly indicates the nucleotide at the assembly location; (3) determining an error value indicating a difference between the count and the reference value; and (4) including the reference value and the error value in the first input. In some embodiments, the system may be configured to determine the reference value based on whether the assembly indicates the nucleotide at the assembly location by: (1) determining the reference value to be a first value (e.g., a number of the plurality of nucleotide sequences) when the assembly indicates the nucleotide at the assembly location; and (2) determining the reference value to be a second value (e.g., 0) when the assembly does not indicate the nucleotide at the assembly location. In some embodiments, the system may be configured to use a neighborhood of 3, 4, 5, 6, 7, 8, 9, 10, 15, 20, 25, 30, 35, 40, 45, or 50 locations.

In some embodiments, the system may be configured to generate the first input to identify a nucleotide at a first assembly location by arranging values into a data structure having rows/column, wherein: (1) a first row/column holds reference values and error values determined for multiple nucleotides at the first assembly location; and (2) a second row/column holds reference values and error values determined for multiple nucleotides at a second location in a neighborhood of the first assembly location.

In some embodiments, the one or more likelihoods that each of the one or more respective biological polymers is present at the assembly location comprises, for each of multiple nucleotides, a likelihood (e.g., probability) that the nucleotide is present at the assembly location. The system may be configured to identify biological polymers at the first plurality of assembly locations in the assembly by identifying a nucleotide at a first one of the first plurality of assembly locations to be a first one of the multiple nucleotides. The system may identify the nucleotide at the first assembly location to be the first nucleotide by determining that a likelihood that the first nucleotide is present at the first assembly location is greater than a likelihood that a second one of the multiple nucleotides is present at the first assembly location.

In some embodiments, the system may be configured to generate the assembly (e.g., an initial assembly) from the plurality of nucleotide sequences. In some embodiments, the system may be configured to generate the assembly by determining a consensus sequence from the plurality of nucleotide sequences (e.g., by taking a majority vote) to be the assembly. In some embodiments, the system may be configured to generate the assembly from the plurality of nucleotide sequences by applying an overlap layout consensus (OLC) algorithm to the plurality of nucleotide sequences. In some embodiments, the system may be configured to: (1) access training data including biological polymer sequences obtained from sequencing a reference macromolecule and a predetermined biological polymer assembly of the reference macromolecule; and (2) train a deep learning model (e.g., a convolutional neural network or recurrent neural network) using the training data to obtain the trained deep learning model. In some embodiments, a reference macromolecule used to train the deep learning model may be different from the macromolecule for which the assembly is being generated.

It should be appreciated that the techniques introduced above and discussed in greater detail below may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

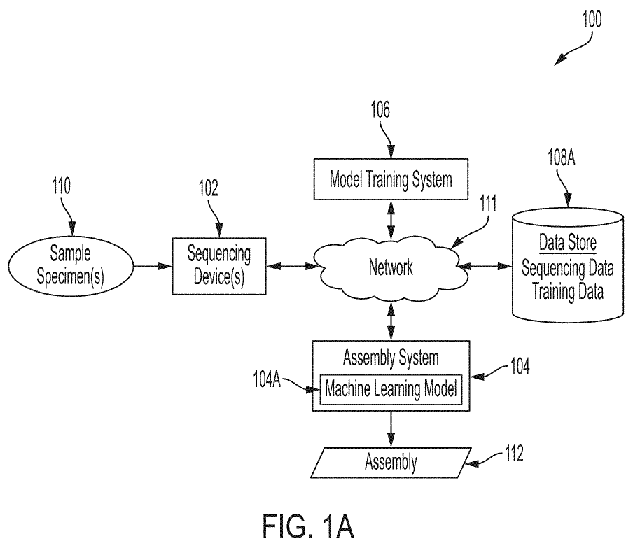

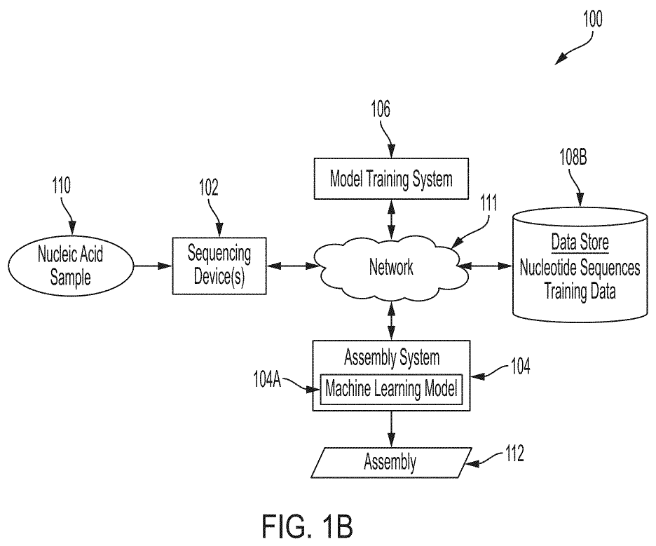

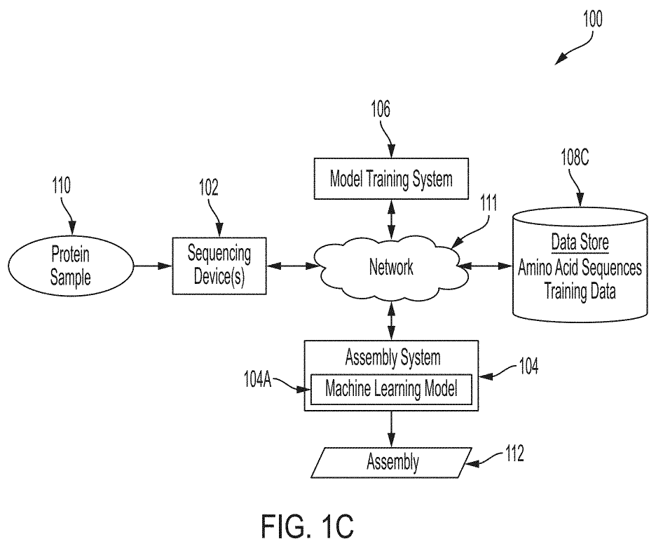

FIG. 1A shows a system 100 in which aspects of the technology described herein may be implemented. The system 100 includes one or more sequencing devices 102, an assembly system 104, a model training system 106, and a data store 108A, each of which is connected to a network 111.

In some embodiments, the sequencing device(s) 102 may be configured to generate sequencing data by sequencing of one or more sample specimens 110 of a macromolecule. For example, the sample specimen(s) 110 may be a biological sample containing nucleic acids (e.g., DNA and/or RNA), or a protein (e.g., a peptide). The sequencing data may include biological polymer sequences of the sample specimen(s) 110. A biological polymer sequence may be represented as a sequence of alphanumeric symbols indicating an order and position of biological polymers present in the macromolecule sample. In some embodiments, the biological polymer sequences may be nucleotide sequences generated from sequencing the biological sample. As an example, a nucleotide sequence may use: (1) “A” to represent adenine; (2) “C” to represent cytosine; (3) “G” to represent guanine; (4) “T” to represent thymine; (5) “U” to represent Uracil; and (6) “-” to represent that no nucleotide is present at a location in the sequence. In some embodiments, the biological polymer sequences may be amino acid sequences generated from sequencing a protein sample (e.g., a peptide). As an example, an amino acid sequence may be an alphanumeric sequence using different alphanumeric characters to represent respective different amino acids that may be present in a protein.

In some embodiments, the sequencing device(s) 102 may be configured to generate nucleotide sequences from sequencing a nucleic acid sample (e.g., DNA sample). In some embodiments, the sequencing device(s) 102 may be configured to sequence the nucleic acid sample by synthesis. The sequencing device(s) 102 may be configured to identify nucleotides as the nucleotides are incorporated into a newly synthesized strand of a nucleic acid that is complementary to the nucleic acid being sequenced. During sequencing, a polymerizing enzyme (e.g., DNA polymerase) may couple (e.g., attach) to a priming location (referred to as a “primer”) of a target nucleic acid molecule and incorporate nucleotides to the primer via the action of the polymerizing enzyme. The sequencing device(s) 102 may be configured to detect each nucleotide that is being incorporated. In some embodiments, the nucleotides may be associated with respective luminescent molecules (e.g., fluorophores) that emit light in response to excitation. A luminescent molecule may be excited when a respective nucleotide that the luminescent molecule is associated with is being incorporated. The sequencing device(s) 102 may include one or more sensors to detect the light emissions. Each type of nucleotide may be associated with a respective type of luminescent molecule. The sequencing device(s) 102 may identify a nucleotide being incorporated by identifying a type of luminescent molecule based on the detected light emissions. For example, the sequencing device(s) 102 may use light emission intensity, lifetime, wavelengths, or other properties to differentiate between different luminescent molecules. In some embodiments, the sequencing device(s) 102 may be configured to detect electrical signals generated during nucleotide incorporation to identify a nucleotide being incorporated. The sequencing device(s) 102 may include sensor(s) to detect electrical signals, and use them to identify nucleotides being incorporated.

In some embodiments, the sequencing device(s) 102 may be configured to sequence a nucleic acid using techniques different from those described herein. Some embodiments are not limited to any particular technique of nucleic acid sequencing described herein.

In some embodiments, the sequencing device(s) 102 may be configured to generate amino acid sequences from sequencing a protein sample (e.g., peptide). In some embodiments, the sequencing device(s) 102 may be configured to sequence the protein sample using reagents that selectively bind to respective amino acids. A reagent may selectively bind to one or more types of amino acids over other types of amino acids. In some embodiments, the reagents may be associated with respective luminescent molecules. The luminescent molecules may be excited in response to an interaction between a reagent that the luminescent molecule is associated with and an amino acid. In some embodiments, the sequencing device(s) 102 may be configured to identify amino acids by detecting light emissions of luminescent molecules. The sequencing device(s) 102 may include one or more sensors to detect the light emissions. In some embodiments, each type of amino acid may be associated with a respective type of luminescent molecule. The sequencing device(s) 102 may identify an amino acid by identifying a type of luminescent molecule based on the detected light emissions. As an example, the sequencing device(s) 102 may use light emission intensity, lifetime, wavelengths, or other properties to differentiate between different luminescent molecules. In some embodiments, the sequencing device(s) 102 may be configured to detect electrical signals generated during binding interactions between reagents and amino acids. The sequencing device(s) 102 may include sensor(s) to detect electrical signals, and use the signals to identify amino acids involved in respective binding interactions.

In some embodiments, the sequencing device(s) 102 may be configured to sequence a protein using techniques different from those described herein. Some embodiments are not limited to any particular technique of protein sequencing described herein.

As illustrated in the embodiment of FIG. 1A, the sequencing device(s) 102 may be configured to transmit sequencing data generated by the device(s) 102 to the data store 108A for storage. The sequencing data may include sequences generated from sequencing of macromolecule samples. The sequencing data may be used by one or more other systems. As an example, the sequencing data may be used by the assembly system 104 to generate an assembly of a macromolecule. As another example, the sequencing data may be used by the model training system 106 as training data to train a machine learning model for use by the assembly system 104. Example uses of sequencing data are described herein.

In some embodiments, the assembly system 104 may be a computing device configured to generate an assembly 112 using sequencing data generated by the sequencing device(s) 102. The assembly system 104 includes a machine learning model 104A that the assembly system 104 uses for generating an assembly. In some embodiments, the machine learning model 104A may be a trained machine learning model obtained from the model training system 106. Examples of machine learning models that may be used by the assembly system 104 are described herein.

In some embodiments, the assembly system 104 may be configured to generate the assembly 112 by updating an initial assembly. The initial assembly may be obtained from application of a conventional assembly algorithm to sequencing data. In some embodiments, the assembly system 104 may be configured to generate the initial assembly. The assembly system 104 may be configured to generate the initial assembly by applying an assembly algorithm to the sequencing data obtained from the sequencing device(s) 102. As an example, the assembly system 104 may apply Overlap Layout Consensus (OLC) assembly or De Bruijn Graph (DBG) assembly to sequencing data (e.g., nucleotide sequences) from the data store 108A to generate the initial assembly. In some embodiments, the assembly system 104 may be configured to obtain an initial assembly generated by a system separate from the assembly system 104. As an example, the assembly system 104 may receive an initial assembly generated by a computing device separate from the assembly system 104 that applied an assembly algorithm to sequencing data generated by the sequencing device(s) 102.

In some embodiments, the assembly system 104 may be configured to update or refine an assembly (e.g., an initial assembly obtained from application of an assembly algorithm) using the trained machine learning model 104A. The assembly system 104 may be configured to update the assembly by correcting one or more errors in the assembly and/or confirming biological polymer indications in the assembly. In some embodiments, the assembly system 104 may be configured to update the assembly by: (1) generating an input to the machine learning model 104A using sequencing data and an assembly; (2) providing the generated input to the machine learning model 104A to obtain a corresponding output; and (3) updating the assembly using the output obtained from the machine learning model 104A. In some embodiments, the output of the machine learning model 104A may indicate, for each of multiple locations in the assembly, one or more likelihoods that each of one or more respective biological polymers (e.g., nucleotides or amino acids) is present at the location in the assembly. As an example, the output may indicate, for each of the locations, probabilities that respective nucleotides are present at the location. In some embodiments, the assembly system 104 may be configured to: (1) identify biological polymers (e.g., nucleotides or amino acids) at assembly locations using the output obtained from the machine learning model 104A; and (2) update the assembly to indicate the identified biological polymers at the locations to obtain the updated assembly. Example techniques for updating an assembly using a machine learning model are described herein.

In some embodiments, the assembly system 104 may be configured to identify locations in an assembly that are to be updated (e.g., corrected or confirmed). The assembly system 104 may be configured to generate input to the machine learning model 104A using the selected locations. In some embodiments, the assembly system 104 may be configured to identify the locations that are to be updated by: (1) determining likelihoods that indications of biological polymers at respective assembly locations are incorrect; and (2) selecting locations that are to be corrected based on the determined likelihoods. In some embodiments, the assembly system 104 may be configured to determine numerical values indicating likelihoods that biological polymers indicated at respective locations are incorrect, and select the locations to be updated based on the likelihood values. As an example, the assembly system 104 may select locations that have a likelihood of being incorrect that is greater than a threshold value.

In some embodiments, the assembly system 104 may be configured to generate inputs to the machine learning model 104A by determining feature values for locations in an assembly. The assembly system 104 may be configured to determine feature values using the assembly and sequences from which the assembly was generated. Example features are described herein. In some embodiments, the assembly system 104 may be configured to generate inputs to the machine learning model 104A for each of multiple locations. For each location, the assembly system 104 may be configured to determine feature values, and provide the feature values as input to the machine learning model 104A to obtain a corresponding output. The assembly system 104 may be configured to use the output corresponding to the input provided for a location to correct a biological polymer indicated at the location, or confirm that the biological polymer indicated at the location is correct. In some embodiments, the multiple locations may be all locations in an assembly. In some embodiments, the multiple locations may be a subset of locations in the assembly.

In embodiments where a subset of locations are updated, the assembly system 104 may be configured to select the subset of locations. The assembly system 104 may be configured to select the subset of locations in a number of ways including: (1) determining likelihoods that the assembly incorrectly indicates biological polymers at multiple locations; and (2) selecting the subset of locations from the multiple locations using the likelihoods. For example, the assembly system 104 may: (1) identify locations having a likelihood that exceeds a threshold likelihood; and (2) select the identified locations to be the subset of locations.

In some embodiments, the assembly system 104 may be configured to generate an input for a location to be corrected using feature values determined at one or more locations in a neighborhood of the location. For a selected location, the machine learning model 104A may utilize context information from surrounding locations in the assembly to generate an output for the selected location. In some embodiments, a neighborhood of a location may include: (1) the selected location; and (2) a set of locations surrounding the selected location. As an example, the neighborhood may be a window of locations centered at the selected location for which the machine learning model 104A is to generate an output. The assembly system 104 may use a window of 5 locations, 10 locations, 15 locations, 20 locations, 25 locations, 30 locations, 35 locations, 40 locations, 45 locations, and/or 50 locations.

In some embodiments, the assembly system 104 may be configured to perform multiple update iterations to generate the final assembly 112. As an example, the assembly system 104 may: (1) perform a first iteration on an initial assembly to obtain a first updated assembly; and (2) perform a second iteration on the first updated assembly to obtain a second updated assembly. In some embodiments, the assembly system 104 may be configured to iteratively perform updates. The assembly system 104 may be configured to perform update iterations until a condition is met. Example conditions are described herein.

In some embodiments, the model training system 106 may be a computing device configured to access the data stored in the data store 108A, and use the accessed data to train a machine learning model for use in generating an assembly. In some embodiments, the model training system 106 may be configured to train a separate machine learning model for different assembly systems. A machine learning model trained for a respective assembly system may be tailored to unique characteristics of the assembly system. As an example, the model training system 106 may be configured to: (1) train a first machine learning model for a first assembly system; and (2) train a second machine learning model for a second assembly system. A separate machine learning model for each of the assembly systems may be tailored to unique error profiles of the respective assembly systems. For example, different assembly systems may employ different assembly algorithms for generating an initial assembly, and the machine learning model trained for each assembly system may be tailored to an error profile of the assembly algorithm.

In some embodiments, the model training system 106 may be configured to provide a single trained machine learning model to multiple assembly systems. As an example, the model training system 106 may aggregate assemblies from multiple assembly systems, and train a single machine learning model. The single machine learning model may be normalized for multiple assembly systems to mitigate model variations resulting from variation in assembly techniques employed by the assembly systems. In some embodiments, the model training system 106 may be configured to provide a single trained machine learning model for multiple sequencing devices. As an example, the model training system 106 may aggregate sequencing data from multiple sequencing devices, and train a single machine learning model. The single machine learning model may be normalized for multiple sequencing devices to mitigate model variations resulting from device variation.

In some embodiments, the model training system 106 may be configured to train a machine learning model by using training data that includes: (1) biological polymer sequences obtained from sequencing one or more reference macromolecules (e.g., DNA, RNA, protein); and (2) one or more predetermined assemblies of the reference macromolecule(s). In some embodiments, the model training system 106 may be configured to use indications of biological polymers in the predetermined assemblies as labels for training the machine learning model. The labels may represent correct or desired indications at the assembly locations. As an example, the training data may include nucleotide sequences from sequencing DNA samples of an organism, and a predetermined genome assembly of the organism. In this example, the model training system 106 may use the indications of nucleotides in the predetermined genome assembly as labels for applying a supervised learning algorithm to the training data.

In some embodiments, the model training system 106 may be configured to access training data from external databases. As an example, the model training system 106 may access: (1) sequencing data from the Pacific Biosciences RS II (Pacbio) database, and/or the Oxford Nanopore MiniION (ONT) database; and (2) predetermined genome assemblies from the National Center for Biotechnology Information (NCBI) database of reference genomes. As another example, the model training system 106 may access protein sequencing data and associated proteome assemblies from the UnitProt database and/or the Human Proteome Project (HPP) database.

In some embodiments, the model training system 106 may be configured to train the machine learning model by applying a supervised learning training algorithm using labelled training data. As an example, the model training system 504 may train a deep learning model (e.g., a neural network) by using stochastic gradient descent. As another example, the model training system 106 may train a support vector machine (SVM) to identify decision boundaries of the SVM by optimizing a cost function. As an example, the model training system 106 may: (1) generate inputs to the machine learning model using sequencing data and an assembly generated from application of an assembly algorithm to the sequencing data; (2) label the inputs using a predetermined assembly of the macromolecule (e.g., from a public database); and (3) apply a supervised training algorithm to the generated inputs and corresponding labels.

In some embodiments, the model training system 106 may be configured to train the machine learning model by applying an unsupervised learning algorithm to training data. As an example, the model training system 106 may identify clusters of a clustering model by performing k-means clustering. In some embodiments, the model training system 106 may be configured to: (1) generate inputs to the machine learning model using sequencing data and an assembly generated from application of an assembly algorithm to the sequencing data; and (2) apply an unsupervised learning algorithm to the generated inputs. As an example, the model training system 106 may train a clustering model where each cluster of the model represents a respective nucleotide, and the cluster classification may indicate a nucleotide at a location in a genome assembly or gene sequence. As another example, the model training system 106 may train a clustering model where each cluster of the model represents a respective amino acid, and the cluster classification may indicate an amino acid at a location in a protein sequence.

In some embodiments, the model training system 106 may be configured to train the machine learning model by applying a semi-supervised learning algorithm to training data. In some embodiments, the model training system 106 may be configured to apply a semi-supervised learning algorithm to training data by: (1) labeling a set of unlabeled training data by applying an unsupervised learning algorithm (e.g., clustering) to training data; and (2) applying a supervised learning algorithm to the labelled training data. As an example, the model training system 106 may: (1) generate inputs to the machine learning model using sequencing data and an assembly generated from application of an assembly algorithm to the sequencing data; (2) apply an unsupervised learning algorithm to the generated inputs to label the inputs; and (3) apply a supervised learning algorithm to the labelled training data.

In some embodiments, the machine learning model may include a deep learning model (e.g., a neural network). In some embodiments, the deep learning model may include a convolutional neural network (CNN). In some embodiments, the deep learning model may include a recurrent neural network (RNN), a multi-layer perceptron, an autoencoder and/or a CTC-fitted neural network model. In some embodiments, the machine learning model may include a clustering model. As an example, the clustering model may include multiple clusters, each of the clusters being associated with a biological polymer (e.g., nucleotides, or amino acids).

In some embodiments, the model training system 106 may be configured to train a separate machine learning model for each of multiple sequencing devices. A machine learning model trained for a respective sequencing device may be tailored to unique characteristics of the sequencing device. As an example, the model training system 106 may: (1) train a first machine learning model for a first sequencing device; and (2) train a second machine learning model for a second sequencing device. A machine learning model trained for a respective sequencing device may be optimized for use with sequencing data generated by the sequencing device. For example, the machine learning model may be optimized for a particular sequencing technology (e.g., third generation sequencing) used by the sequencing device.

In some embodiments, the model training system 106 may be configured to periodically update a previously trained machine learning model. In some embodiments, the model training system 106 may be configured to update a previously trained model by updating values of one or more parameters of the machine learning model using new training data. In some embodiments, the model training system 106 may be configured update the machine learning model by training a new machine learning model using a combination of previously-obtained training data and new training data.

In some embodiments, the model training system 106 may be configured to update a machine learning model in response to any one of different types of events. For example, in some embodiments, the model training system 106 may be configured to update the machine learning model in response to a user command. As an example, the model training system 106 may provide a user interface via which the user may command performance of a training process. In some embodiments, the model training system 106 may be configured to update the machine learning model automatically (i.e., not in response to a user command), for example, in response to a software command. As another example, in some embodiments, the model training system 106 may be configured to update the machine learning model in response to detecting one or more conditions. For example, the model training system 106 may update the machine learning model in response to detecting expiration of a period of time. As another example, the model training system 106 may update the machine learning model in response to receiving a threshold amount (e.g., number of sequences and/or assemblies) of new training data.

Although in the example embodiment illustrated in FIG. 1A the model training system 106 is separate from the assembly system 104, in some embodiments, the model training system 106 may be part of the assembly system 104. Although in the example embodiment illustrated in FIG. 1A the assembly system 104 is separate from the sequencing device(s) 102, in some embodiments, the assembly system 104 may be a component of a sequencing device. In some embodiments, the sequencing device 102, model training system 106, and assembly system 104 may each be components of a single system.

In some embodiments, the data store 108A may be a system for storing data. In some embodiments, the data store 108A may include one or more databases hosted by one or more computing devices (e.g., servers). In some embodiments, the data store 108A may include one or more physical storage devices. As an example, the physical storage device(s) may include one or more solid state drives, hard disk drives, flash drives, and/or optical drives. In some embodiments, the data store 108A may include one or more files storing data. As an example, the data store 108A may include one or more text files storing data. As another example, the data store 108A may include one or more XML files. In some embodiments, the data store 108A may be storage (e.g., a hard drive) of a computing device. In some embodiments, the data store 108A may be a cloud storage system.

In some embodiments, the network 111 may be a wireless network, a wired network, or any suitable combination thereof. As one example, the network 111 may be a Wide Area Network (WAN), such as the Internet. In some embodiments, the network 111 may be a local area network (LAN). The local area network may be formed by wired and/or wireless connections between the sequencing device(s) 102, assembly system 104, model training system 106, and the data store 108A. Some embodiments are not limited to any particular type of network described herein.

FIG. 1B shows an example of the system 100 when configured for generating a gene assembly. A gene assembly may be a genome assembly, or a gene sequence. For example, the outputted assembly 112 may be a gene assembly. The sequencing device(s) 102 may be configured to sequence a nucleic acid sample 110 to generate nucleotide sequences. As an example, the sequencing device(s) 102 may sequence a DNA sample from an organism to generate the nucleotide sequences. The nucleotide sequences generated by the sequencing device(s) 102 may be stored in the data store 108B. The assembly system 104 may be configured to use the machine learning model 104A to generate the gene assembly. As an example, the assembly system 104 may: (1) obtain an initial gene assembly by applying an assembly technique (e.g., OLC) to nucleotide sequences generated by the sequencing device(s) 102; and (2) update the initial gene assembly using the machine learning model 104A to obtain the gene assembly 112.

FIG. 1C shows an example of the system 100 when configured for generating a protein sequence. For example, the outputted assembly 112 may be a protein sequence. The sequencing device(s) 102 may be configured to sequence a protein sample 110 to generate amino acid sequences. As an example, the sequencing device(s) 102 may sequence peptides from a protein to generate the amino acid sequences. The amino acid sequences generated by the sequencing device(s) 102 may be stored in the data store 108C. The assembly system 104 may be configured to use the machine learning model 104A to generate the protein sequence. As an example, the protein sequencing system 104 may: (1) obtain a protein sequence by applying an assembly algorithm to amino acid sequences generated by the sequencing device(s) 102; and (2) update the protein sequence using the machine learning model 104A to obtain the protein sequence.

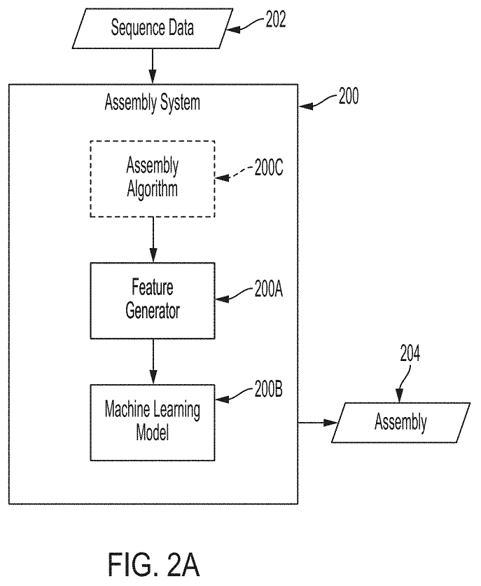

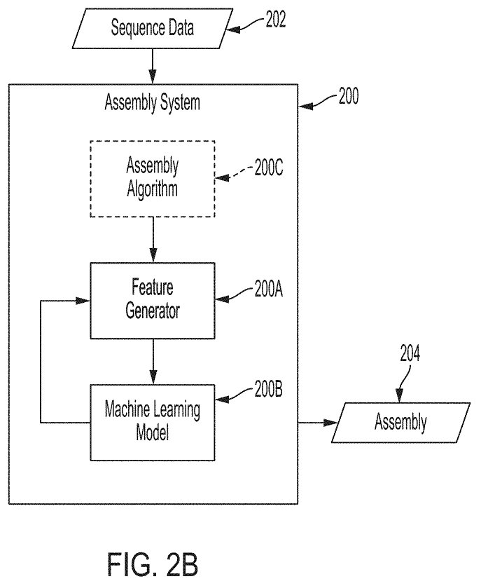

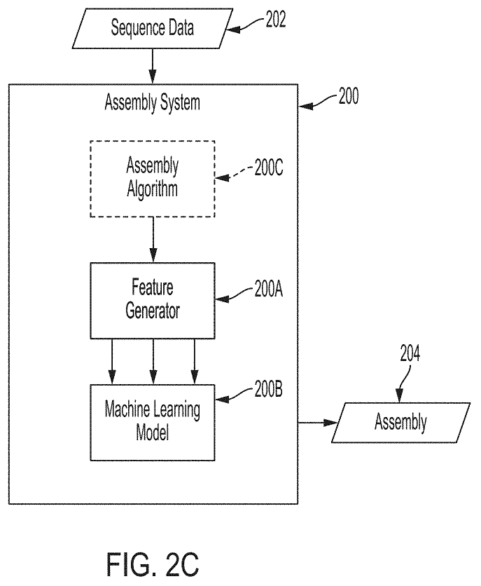

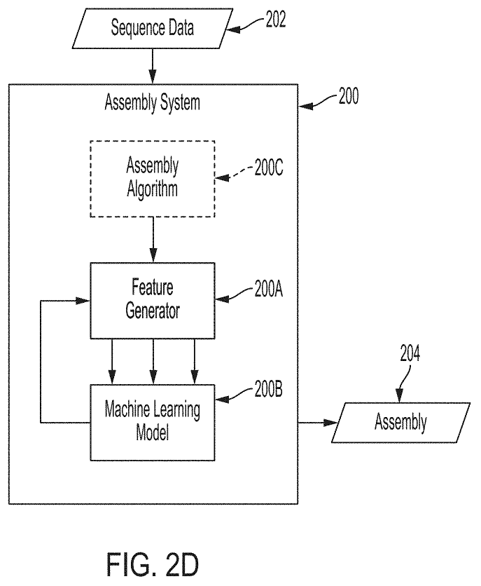

FIG. 2A shows an assembly system 200 for generating an assembly, in accordance with some embodiments of the technology described herein. The assembly system 200 may be assembly system 104 described above with reference to FIGS. 1A-C. Assembly system 200 may be a computing device configured to generate an assembly 204 using sequencing data 202. The assembly system 200 includes multiple components including a feature generator 200A and a machine learning model 200B. The assembly system 200C may optionally include an assembler 200C.

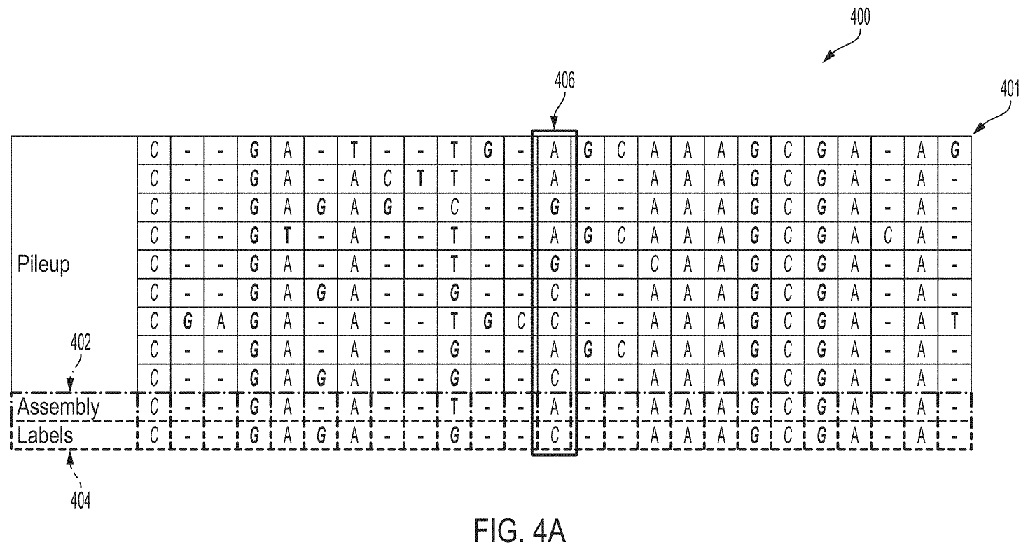

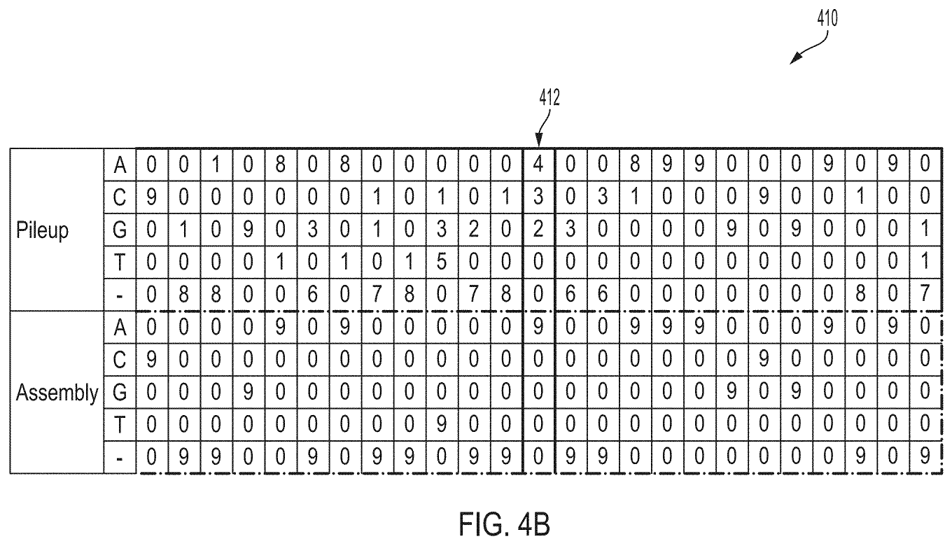

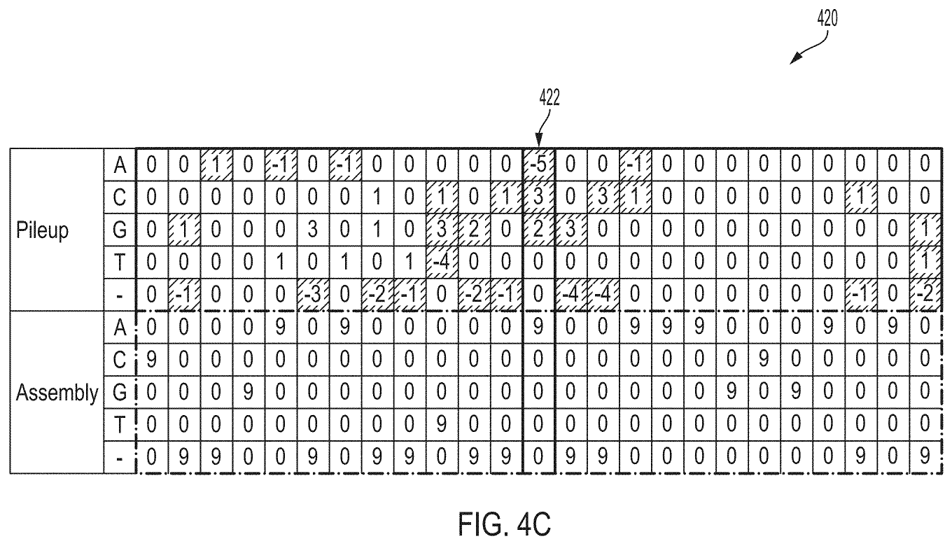

In some embodiments, the feature generator 200A may be configured to determine values of one or more features that may be provided as input to a machine learning model. The feature generator 200A may be configured to determine values of the feature(s) from: (1) sequence data 202; and (2) an assembly (e.g., obtained from application of an assembly algorithm to the sequence data 202). The sequence data 202 may include multiple sequences which are used by the assembly algorithm to generate the assembly. In some embodiments, the feature generator 200A may be configured to determine the values of the feature(s) by comparing each of the sequences to the assembly. In some embodiments, the feature generator 200A may be configured to align the sequences with a portion of the assembly. For example, the feature generator 200A may align the sequences with a set of locations in the assembly where the biological polymers indications at the set of locations in the assembly were determined from the aligned sequences. The feature generator 200A may be configured to determine values of the feature(s) by comparing the aligned sequences to biological polymers (e.g., nucleotides, amino acids) indicated at the set of locations in the assembly. Example techniques for determining values of the feature(s) are described below in reference to FIGS. 4A-C.

As illustrated in the embodiment of FIG. 2A, the feature generator 200A may be configured to generate input to be provided to the machine learning model 200B. In some embodiments, the feature generator 200A may be configured to generate an input for each of multiple locations in an assembly. In some embodiments, the feature generator 200A may be configured to select the locations, and generate the input using the selected locations. In some embodiments, the feature generator 200A may be configured to select the locations by determining likelihoods that the assembly incorrectly indicates biological polymers at the locations, and selecting the locations using the determined likelihoods. In some embodiments, the feature generator 200A may be configured to determine a likelihood that the assembly incorrectly indicates a biological polymer at a location based on a number of sequences aligned with the location that specify a different biological polymer at the location than a biological polymer indicated in the assembly. The feature generator 200A may be configured to generate an input for the location when it is determined that the likelihood exceeds a threshold likelihood.

In some embodiments, the feature generator 200A may be configured to generate an input to be provided to the machine learning model 200B for a target location in an assembly using: (1) a biological polymer identified at the target location; and (2) biological polymers identified at one or more other locations in a neighborhood of the target location. In some embodiments, the feature generator 200A may be configured to determine feature values at the target location and at the other location(s) that are in the neighborhood of the target location. The feature values at the other location(s) in the neighborhood may provide contextual information to the machine learning model 200A to generate an output for the target location. In some embodiments, a size of the neighborhood may be a configurable parameter. For example, the size of the neighborhood may be specified by a user input in a software application.

In some embodiments, the feature generator 200A may be configured to generate an input as a window including feature values determined at locations in a neighborhood of the target location. The neighborhood of the target location may include the target location and one or more other locations in a window of the target location. In some embodiments, the size of the window may be 2 locations, 3 locations, 5 locations, 10 locations, 15 locations, 20 locations, 25 locations, 30 locations, 35 locations, 40 locations, 45 locations, or 50 locations. In some embodiments, the feature generator 200A may be configured to use a neighborhood size 60 locations, 70 locations, 80 locations, 90 locations, or 100 locations. In some embodiments, the window may be centered at the target location.

In some embodiments, the machine learning model 200B may be machine learning model 104A described above with reference to FIGS. 1A-C. As illustrated in the embodiment of FIG. 1A, the machine learning model 200B may be configured to receive input from the feature generator 200A. The machine learning model 200B may be configured to generate an output corresponding to a respective input provided by the feature generator 200A. The machine learning model 200B may be configured to generate an output that is used by the assembly system 200 to identify biological polymers (e.g., nucleotides or amino acids) at locations in the assembly. In some embodiments, the machine learning model 200B may be configured to output, for a location, likelihoods that each of multiple biological polymers is present at the location. As an example, the machine learning model 200B may output, for each of multiple nucleotides, a probability that the nucleotide is present at the location. As another example, the machine learning model 200B may output, for each of multiple amino acids, a probability that the amino acid is present at the location. In some embodiments, the assembly system 200 may be configured to identify a biological polymer at a location in the assembly to be a biological polymer having the greatest likelihood of being present at the location of the biological polymers as indicated by the output of the machine learning model 200B. As an example, the assembly system 200 may select, from among multiple nucleotides, the one that has the greatest probability of being present at the location. As another example, the assembly system 200 may select, from among multiple amino acids, the one that has the greatest probability of being present at the location.

In some embodiments, the assembly system 200 may be configured to use the output obtained from the machine learning model 200B to generate the output assembly 204. The assembly system 200 may be configured to update the assembly using the biological polymers identified at locations in the assembly from the output obtained from the machine learning model 200B. The assembly system 200 may be configured to update the assembly to indicate the identified biological polymers at the locations in the assembly to obtain the output assembly 204. As one example, an assembly may indicate adenine at a first location in the assembly and guanine at a second location in the assembly. In this example, the assembly system 200 may: (1) identify a nucleotide at the first location to be thymine, and a nucleotide at the second location to be guanine using an output obtained from the machine learning model 200B; and (2) update the first location in the assembly to indicate thymine, and leave the indicated nucleotide at the second location unchanged to generate the output assembly 204. As illustrated by the above example, the assembly system 200 may modify biological polymer indications at location(s) in the assembly using the output obtained from the machine learning model 200B while leaving biological polymer indications at other location(s) unchanged. For example, the assembly system 200 may determine that a biological polymer identified at a location in the assembly matches a biological polymer indicated in the assembly and leave the indication at the location unchanged in the updated assembly.

As shown in the embodiment of FIG. 1A, the assembler 200C may be configured to provide an assembly to the feature generator 200A. In some embodiments, the assembler 200C may be configured to generate an assembly to be provided to the feature generator 200A by applying an assembly algorithm to sequence data 202 (e.g., received from sequencing a macromolecule sample). As an example, the assembler 200C may be configured to apply an assembly algorithm to nucleotide sequences included in the sequence data 202 to generate the assembly. The assembly may then be provided to the feature generator 200A to generate input to be provided to the machine learning model 200B to obtain output for identifying biological polymers at locations in the assembly. The assembly generated by the assembler 200C may be updated by the assembly system 200 using the output obtained from the machine learning model 200B to generate the output assembly 204.

In some embodiments, the assembler 200C may be configured to apply an overlay layout consensus (OLC) algorithm to nucleotide sequences included in the sequence data 202 to generate an assembly. A sequencing device may sequence multiple copies of a biological sample including nucleic acid(s). As a result, the sequence data 202 may include, for each portion (e.g., set of locations) of an assembly, multiple sequences that align to the portion of the assembly. A mean number of sequences that cover a location in the assembly may be referred to as “coverage” of the sequences. The assembler 200C may be configured to apply the OLC algorithm to the sequences by: (1) generating an overlap graph based on overlapping regions of the sequences; (2) using the overlap graph to generate a layout of sequences (also referred to as “contigs”) that align with respective portions of an assembly; and (3) for each set of sequences that align to a portion of the assembly, taking a consensus of the sequences in the set to generate the portion of the assembly.

In some embodiments, the assembler 200C may be configured to identify sequences that have overlapping regions by comparing pairs of sequences to determine if they include one or more identical subsequences of biological polymers (e.g., nucleotides). In some embodiments, the assembler 200C may be configured to: (1) identify pairs of sequences that share identical subsequence(s) of at least a threshold number (e.g., 3, 4, 5, 6, 8, 10, 20, 30, 40, 50, 60, 70, 80, 90, 100, 200, 300, 400, 500) of nucleotides to be overlapping sequences; (2) determine the length (i.e., number of nucleotides) of each overlapping region; and (3) generate an overlap graph based on the identified overlapping sequences and lengths of the overlapping regions. The overlap graph may include sequences as vertices and edges connecting respective pairs of sequences that overlap. The determined lengths may be used as labels of the edges in the overlap graph.

In some embodiments, the assembler 200C may be configured to generate a layout of sets of sequences aligned with respective portions of an assembly by concatenating sequences together using an overlap graph. The assembler 200C may be configured to find paths through the overlap graph to concatenate the sequences. As an example, the assembler 200C may concatenate a set of alphanumeric characters representing nucleotides to obtain the concatenated sequences. In some embodiments, the assembler 200C may apply a greedy algorithm to the overlap graph to identify the concatenated sequences. As an example, the assembler 200C may apply a greedy algorithm to identify a shortest common superstring as the concatenated sequences.

In some embodiments, the assembler 200C may be configured to use the layout sequences to generate the assembly. In some embodiments, the assembler 200C may identify multiple sets of layout sequences, where each set aligns with a portion of the assembly. The assembler 200C may be configured to generate the portion of the assembly by taking a consensus of the layout sequences that align with the portion of the assembly. In some embodiments, the assembler 200C may be configured to take a consensus by determining a biological polymer (e.g., a nucleotide) at a location in the portion of the assembly to be a biological polymer that a majority of the sequences aligned to the portion of the assembly indicate is at the location. As an example, the assembler 200C may generate an overlap graph of the nucleotide sequences, and identify four nucleotide sequences “TAGA,” “TAGA,” “TAGT,” “TAGA,” and “TAGC” that correspond to a set of four locations in an assembly. In this example, the assembler 200C may determine a consensus among the four nucleotide sequences to be “TAGA” as all four of the nucleotide sequences indicate the first three locations to be “TAG”, and a majority of the nucleotide sequences indicate the fourth location to be “A.”

In some embodiments, the assembly system 200 may be configured to perform a consensus step of the OLC algorithm using machine learning techniques. When the assembler 200C has generated a layout to be used for generating an assembly, the system may be configured to use the layout and consensus assembly obtained from the layout to generate input to the machine learning model. In some embodiments, the assembly system 200 may be configured to update the consensus assembly using techniques described herein to obtain the output assembly 204.

In some embodiments, the assembler 200C may be configured to apply an algorithm to sequence data 202 described in “Assembly Algorithms for Next-Generation Sequencing Data,” published in Genomics Volume 95, Issue 6, June 2010, which is incorporated herein by reference. In some embodiments, the assembler 200C may be configured to apply an assembly algorithm other than an OLC algorithm to the sequence data 202 to generate an assembly. In some embodiments, the assembler 200C may be configured to apply de Bruijn graph (DBG) assembly to the sequence data 202. Some embodiments are not limited to a particular type of assembly algorithm. In some embodiments, the assembler 200C may include a software application configured to generate an assembly using sequence data 202. As an example, the system may include the HGAP, Falcon, Canu, Hinge, Miniasm, or Flye assembler. As another example, the system may include the SPAdes, Ray, ABySS, ALLPATHS-LG, or Trinity assembly application. Some embodiments are not limited to a particular assembler.

As indicated by the dashed lines in FIG. 2A, in some embodiments, the assembler 200C may not be included in the assembly system. The assembly system 200 may be configured to receive an assembly from a separate system and update the received assembly to generate the output assembly 204. As an example, a separate computing device may apply an assembly algorithm (e.g., OLC) to the sequence data 202 to generate an assembly, and transmit the generated assembly to the assembly system 200.

FIG. 2B shows an embodiment of the assembly system 200 described above with reference to FIG. 2A in which the assembly system 200 is configured to perform multiple iterations of updates to an assembly, as indicated by the feedback arrow from the machine learning model 200B to the feature generator 200A. In some embodiments, the assembly system 200 may be configured to determine values of one or more features that may be provided as input to the machine learning model 200B after obtaining a first updated assembly. The feature generator 200A may be configured to determine values of feature(s) from: (1) sequence data 202; and (2) the first updated assembly obtained from updating an initial assembly obtained from application of an assembly algorithm to the sequence data 202. The feature generator 200A may be configured to provide the determined values of the feature(s) as input to the machine learning model 200B to obtain an output. The assembly system 200 may be configured to use the output from the machine learning model 200B to: (1) identify biological polymers at respective locations in the first updated assembly; and (2) update the first updated assembly to indicate the identified biological polymers at the respective locations to obtain a second updated assembly. The second updated assembly may be assembly 204 output by the assembly system 200.

In some embodiments, the assembly system 200 may be configured to perform update iterations until a condition is met. In some embodiments, the assembly system 104 may be configured to perform update iterations until the system determines that a threshold number of iterations have been performed. In some embodiments, the threshold number of iterations may be set by a user input (e.g., a software command, or hard-coded value). In some embodiments, the assembly system 104 may be configured to determine a threshold number of iterations. As an example, the assembly system 200 may determine a threshold number of update iterations based on a type of assembly technique that was used to obtain an initial assembly. In some embodiments, the assembly system 200 may be configured to iteratively update the assembly until a specified stopping criterion has been satisfied. As an example, the assembly system 200 may: (1) determine a number of differences between a current assembly obtained from the latest update iteration and a previous assembly; and (2) determine to stop iteratively updating the assembly when the number of differences is less than a threshold number of differences and/or when a percentage of differences is less than a threshold percentage.

FIG. 2C shows an embodiment of the assembly system 200 described above with reference to FIG. 2A in which the assembly system 200 is configured to correct multiple locations of an assembly in parallel, as indicated by the multiple arrows from feature generator 200A to the machine learning model 200B. As described in reference to FIG. 2A, in some embodiments, the feature generator 200A may be configured to generate input to be provided to the machine learning model 200B for each of multiple locations. In the embodiment of FIG. 2C, the assembly system 200 may be configured to update multiple locations of an assembly in parallel. The assembly system 200 may be configured to: (1) update a first location in the assembly; and (2) prior to completing an update of the first location in the assembly, begin updating a second location in the assembly. In some embodiments, the assembly system 200 may be configured to update multiple locations in parallel by generating and/or providing multiple inputs generated for multiple respective locations to the machine learning model 200B in parallel. As an example, the feature generator 200A may: (1) generate and/or provide a first input for a first location to the machine learning model 200B; and (2) prior to obtaining an output from the machine learning model 200B corresponding to the first input, generate and/or provide a second input for a second location to the machine learning model 200B.

In some embodiments, the assembly system 200 of FIG. 2C may be a computing device that includes multiple processors configured to update multiple locations of an assembly in parallel. In some embodiments, the assembly system 200 may be configured to use a multi-threaded application where each thread of the application is configured to update a respective location in an assembly in parallel with one or more other threads.

FIG. 2D shows an embodiment of the assembly system 200 described above with reference to FIG. 2A in which the assembly system 200 is configured to: (1) perform multiple iterations of updates, as indicated by the arrow from the machine learning model 200B to the feature generator 200A; and (2) correct multiple locations of an assembly in parallel, as indicated by the multiple arrows from feature generator 200A to the machine learning model 200B. In some embodiments, the assembly system 200 may be configured to perform multiple update iterations as described above with reference to FIG. 2B and, during each update cycle, update multiple locations in an assembly in parallel as described above with reference to FIG. 2C.

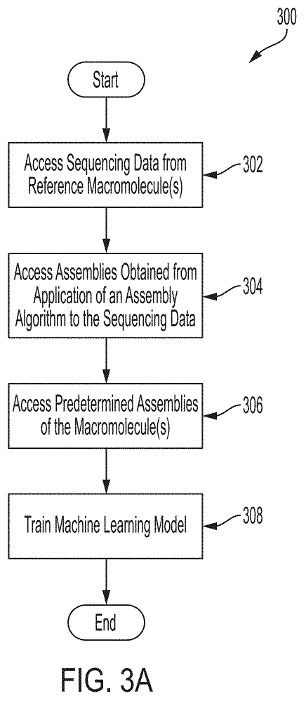

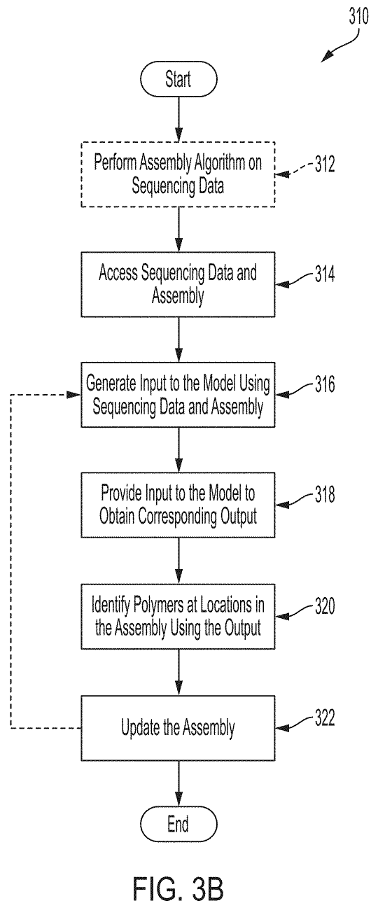

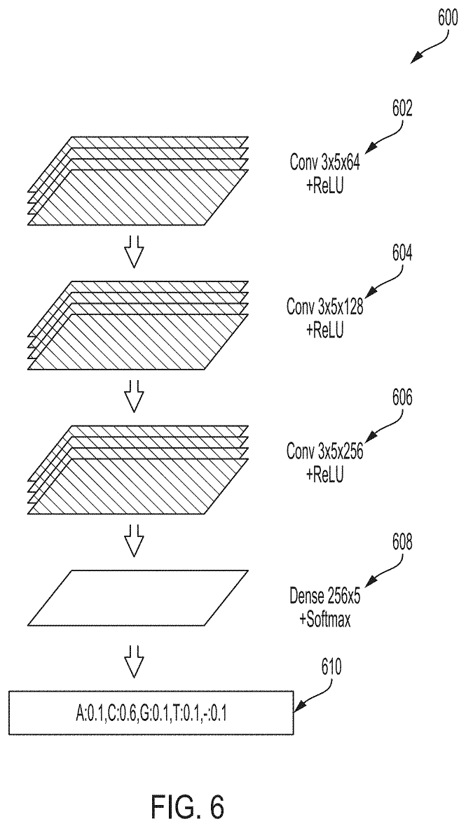

FIG. 3A illustrates an example process 300 for training a machine learning model for generating a biological polymer assembly, according to some embodiments of the technology described herein. Process 300 may be performed by any suitable computing device(s). As an example, process 300 may be performed by model training system 106 described with reference to FIGS. 1A-C. Process 300 may be performed to train machine learning models described herein. As an example, process 300 may be performed to train a deep learning model such as convolutional neural network (CNN) 600 described with reference to FIG. 6.

In some embodiments, the machine learning model may be a deep learning model. In some embodiments, the deep learning model may be a neural network. As an example, the machine learning model may be a convolutional neural network (CNN) that generates an output for use in identifying biological polymers (e.g., nucleotides, amino acids) at locations in an assembly. As another example, the machine learning model may be a CTC-fitted neural network. In some embodiments, portions of the deep learning model may be trained separately. As an example, the deep learning model may have a first portion which encodes input data in values of one or more features, and a second portion which receives the values of the feature(s) as input to generate an output identifying one or more biological polymers.

In some embodiments, the machine learning model may be a clustering model. In some embodiments, each cluster of the model may be associated with a biological polymer. As an illustrative example, the clustering model may include 5 clusters, where each cluster is associated with a respective nucleotide. For example, the first cluster may be associated adenine; the second cluster may be associated with cytosine; the third cluster may be associated with guanine; the fourth cluster may be associated with thymine; and the fifth cluster may indicate that no nucleotide is present (e.g., at a location in an assembly). Example numbers of clusters and associated biological polymers are described herein for illustrative purposes.

Process 300 begins at block 302, where the system executing process 300 accesses sequencing data from sequencing one or more reference macromolecules (e.g., DNA, RNA, or proteins). In some embodiments, the system may be configured to access sequencing data from sequencing reference macromolecules from a database. As an example, the system may access sequencing data obtained from sequencing of bacteria from the ONG database. The sequencing data may be obtained from sequencing one or more samples of a macromolecule. As an example, the sequencing data may be obtained from biological samples of Saccharomyces cerevisiae, which is a species of yeast. As another example, the sequencing data may be obtained from sequencing peptide samples of a protein. In some embodiments, the sequencing data may include nucleotide sequences obtained from sequencing biological samples including a nucleic acid (e.g., DNA, RNA). In some embodiments, the sequencing data may include amino acid sequences obtained from sequencing protein samples (e.g., peptides from the protein).

In some embodiments, the system may be configured to access sequencing data from a target sequencing technology such that the machine learning model may be trained to improve accuracy of assemblies generated from sequencing data generated by the target sequencing technology. The machine learning model may be trained for an error profile of the target sequencing technology such that the machine learning model may be optimized to correct errors characteristic of the target sequencing technology. In some embodiments, the system may be configured to access data obtained from third generation sequencing. In some embodiments, the third generation sequencing may be single-molecule real-time sequencing. As an example, the system may access data obtained from a system that sequences nucleic acid samples by detecting light emissions by luminescent molecules associated with nucleotides. As another example, the system may access data obtained from a system that sequences peptides by detecting light emissions by luminescent molecules associated with reagents that selectively interact with amino acids. In some embodiments, the system may be configured to access data obtained from second generation sequencing. As an example, the system may access sequencing data obtained from Sanger sequencing, Maxam-Gilbert sequencing, shotgun sequencing, pyrosequencing, combinatorial probe anchor synthesis, or sequencing by ligation. In some embodiments, the system may be configured to access data obtained from de novo peptide sequencing. As an example, the system may access amino acid sequences obtained from tandem mass spectrometry. Some embodiments are not limited to a particular target sequencing technology.

Next, process 300 proceeds to block 304, where the system accesses assemblies generated from at least a portion of the sequencing data obtained at block 302. In some embodiments, the system may be configured to access assemblies obtained from application of an assembly algorithm (e.g., OLC assembly, DBG assembly) to the sequencing data. In some embodiments, the system may be configured to access the assemblies by applying an assembly algorithm to the sequencing data. In some embodiments, the system may be configured to access pre-determined assemblies generated from application of one or more assembly algorithms to the sequencing data. As an example, the assemblies may have been previously performed by a separate computing device and stored in a database. For example, a database from which the sequencing data was obtained may also store assemblies generated from application of one or more assembly algorithms to the sequencing data.

In some embodiments, the system may be configured to access assemblies generated from a target assembly technology, such that the machine learning model may be trained to correct errors that are characteristic to the target assembly technology. The machine learning model may be trained for an error profile of the target assembly technology such that the machine learning model may be optimized to correct errors characteristic of the target assembly technology. In some embodiments, the system may be configured to access assemblies generated by a particular assembly algorithm and/or software application. As an example, the system may access assemblies generated by the Canu, Miniasm, or Flye assembler. In some embodiments, the system may be configured to access assemblies generated from a class of assemblers. As an example, the system may access assemblies generated from greedy algorithms assemblers, or graph method assemblers. Some embodiments are not limited to a particular assembly technology.

Next, process 300 proceeds to block 306, where the system access one or more predetermined assemblies of the reference macromolecule(s). In some embodiments, the predetermined assemblies of the reference macromolecule(s) may represent true or correct assemblies for respective macromolecule(s). As such, the system may be configured to use the predetermined assemblies of the reference macromolecule(s) to label training data. As an example, the system may access a reference genome of an organism's DNA from the NCBI database. In this example, the system may use the reference genome to determine labels for use in performing supervised learning to train a machine learning model for identifying nucleotides in a genome assembly. As another example, the system may access a reference protein sequence of a protein from the UnitProt database, and use the reference protein sequence to determine labels for use in performing supervised learning to train a machine learning model for identifying amino acids in a protein sequence.

Next, process 300 proceeds to block 308 where the system trains a machine learning model using the data accessed at blocks 302-308. In some embodiments, the system may be configured to: (1) generate inputs to the machine learning model using the sequencing data accessed at block 302 and the assemblies accessed at block 304; (2) label the generated inputs using the predetermined assemblies accessed at block 306; and (3) apply a supervised learning algorithm to the labelled training data. In some embodiments, the system may be configured to generate inputs to the machine learning model by generating values of one or more features using the sequencing data. In some embodiments, the system may be configured to determine values of feature(s) for each location in an assembly. As an example, the system may determine values of features for a location by: (1) determining counts for respective nucleotides, where each count indicates a number of nucleotide sequences that indicate that the nucleotide is present at the location; and (2) determine the values of the feature(s) using the counts. Example techniques for generating inputs and labelling the inputs are described herein with reference to FIGS. 4A-C.

In some embodiments, the system may be configured to train a deep learning model using the labeled training data. In some embodiments, the system may be configured to train a decision tree model using the labelled training data. In some embodiments, the system may be configured to train a support vector machine (SVM) using the labelled training data. In some embodiments, the system may be configured to train a Naïve Bayes classifier (NBC) using the labelled training data.

In some embodiments, the system may be configured to train the machine learning model by using stochastic gradient descent. The system may make changes to parameters of the machine learning model iteratively to optimize an objective function to obtain a trained machine learning model. For example, the system may use stochastic gradient descent to train filters of a convolutional network and/or weights of a neural network.

In some embodiments, the system may be configured to perform supervised training using the labeled training data. In some embodiments, the system may be configured to train the machine learning model by: (1) providing the generated inputs to the machine learning model to obtain corresponding outputs; (2) identifying biological polymers that are present at locations in the assembly using the outputs; and (2) training the machine learning model based on a difference between the identified biological polymers and biological polymers indicated at the locations in the reference assemblies. A biological polymer indicated at a location in a reference assembly may be a label for a respective input. The difference may provide a measure of how well the machine learning model performs in reproducing the label when configured with its current set of parameters. As an example, the parameters of the machine learning model may be updated using stochastic gradient descent and/or any other iterative optimization technique suitable for training the model. As an example, the system may be configured to update one or more parameters of the model based on the determined difference.

In some embodiments, the system may apply an unsupervised training algorithms to a set of unlabeled training data. Although the embodiment of FIG. 3A includes accessing a predetermined assemblies of reference macromolecules at block 306, in some embodiments, the system may be configured to perform training without accessing predetermined assemblies. In these embodiments, the system may be configured to apply an unsupervised training algorithm to training data to train the machine learning model. The system may be configured to train the model by: (1) generating inputs to the model using the sequencing data and assemblies generated from the sequencing data; and (2) apply an unsupervised training algorithm to the generated inputs. In some embodiments, the machine learning model may be a clustering model and the system may be configured to identify clusters of the clustering model by applying an unsupervised learning algorithm to training data. Each cluster may be associated with a biological polymer (e.g., nucleotide or amino acid). As an example, the system may perform k-means clustering to identify clusters (e.g., cluster centroids) using the training data.

In some embodiments, the system may be configured to apply a semi-supervised learning algorithm to training data. The system may: (1) label a set of unlabeled training data by applying an unsupervised learning algorithm (e.g., clustering) to training data; and (2) applying a supervised learning algorithm to the labelled training data. As an example, the system may apply k-means clustering to inputs generated from the sequencing data and the assemblies obtained from the sequencing data to cluster the inputs. The system may then label each input with a classification based on cluster membership. The system may then train the machine learning model by applying a stochastic gradient descent algorithm and/or any other iterative optimization technique to the labelled data.

After training the machine learning model at block 308, process 300 ends. In some embodiments, the system may be configure to store the trained machine learning model. The system may store value(s) of one or more trained parameters of the machine learning model. As an example, the machine learning model may include one or more neural networks and the system may store values of trained weights of the neural network(s). As another example, the machine learning model include a convolutional neural network and the system may store one or more trained filters of the convolutional neural network. In some embodiments, the system may be configured to store the trained machine learning model (e.g., in assembly system 104) for use in generating an assembly (e.g., a genome assembly, a protein sequence, or portion thereof).

In some embodiments, the system may be configured to obtain new data to update the machine learning model using new training data. In some embodiments, the system may be configured to update the machine learning model by training a new machine learning model using the new training data. As an example, the system may train a new machine learning model using the new training data. In some embodiments, the system may be configured to update the machine learning model by retraining the machine learning model using the new training data to update one or more parameters of the machine learning model. As an example, the output(s) generated by the model and corresponding input data may be used as training data along with previously obtained training data. In some embodiments, the system may be configured to iteratively update the trained machine learning model using data and outputs identifying amino acids (e.g., obtained from performing process 310 described below in reference to FIG. 3B). As an example, the system may be configured to provide input data to a first trained machine learning model (e.g., a teacher model), and obtain an output identifying one or more amino acids. The system may then retrain the machine learning model using the input data and the corresponding output to obtain a second trained machine learning model (e.g., a student model).

In some embodiments, the system may be configured to train a separate machine learning model for each of multiple sequencing technologies. A machine learning model may be trained for a respective sequencing technology using data obtained from the sequencing technology. The machine learning model may be tuned for an error profile of the sequencing technology. In some embodiments, the system may be configured to train a separate machine learning model for each of multiple assembly technologies. A machine learning model may be trained for a respective assembly technology using assemblies obtained from the assembly technology. The machine learning model may be tuned for an error profile of the assembly technology.

In some embodiments, the system may be configured to train a generalized machine learning model that is to be used for multiple sequencing technologies. The generalized machine learning model may be trained using data aggregated from multiple sequencing technologies. In some embodiments, the system may be configured to train a generalized machine learning model that is to be used for multiple assembly technologies. The generalized machine learning model may be trained using assemblies generated using the multiple assembly technologies.

FIG. 3B illustrates an example process 310 for using a trained machine learning model obtained from process 300 for generating an assembly (e.g., genome assembly, gene sequence, protein sequence, or portion thereof), according to some embodiments of the technology described herein. Process 310 may be performed by any suitable computing device. As an example, process 310 may be performed by assembly system 104 described above with reference to FIGS. 1A-C.

Process 310 begins at block 312 where the system performs an assembly algorithm (e.g., OLC assembly, or DBG assembly) on sequencing data to generate an assembly. As an example, the system may apply an assembly algorithm to nucleotide sequences generated from sequencing of a DNA sample. As another example, the system may apply an assembly algorithm to amino acid sequences generated from sequencing of a peptide sample from a protein. The system may apply an assembly algorithm as described above with reference to assembler 200C of FIGS. 2A-D. In some embodiments, the system may include an assembly application. The system may be configured to generate the assembly by executing the assembly application. Examples of assembly applications are described herein.

As illustrated by the dashed lines around block 312, in some embodiments, the system may not perform an assembly algorithm. The system may obtain an assembly generated by a separate system (e.g., a separate computing device), and perform the steps of block 314-322 to update the obtained assembly.

Next, process 310 proceeds to block 312 where the system accesses sequencing data and an assembly. In some embodiments, the system may be configured to access an assembly generated by the system (e.g., at block 312). In some embodiments, the system may be configured to access an assembly generated by a separate system. As an example, the system may receive an assembly generated by a software application executing on a computing device separate from the system. In some embodiments, the system may be configured to access sequencing data generated from a target assembly technology (e.g., algorithm and/or software application) that the machine learning model trained in process 300 has been optimized to update (e.g., to correct errors). As an example, the machine learning model may be trained on assemblies generated from the Canu assembly application, and the system may access an assembly generated by the Canu assembly application.

In some embodiments, the system may be configured to access sequencing data that includes biological polymer sequences that were used to generate the accessed assembly. As an example, the accessed sequencing data may include nucleotide sequences on which an assembly algorithm was applied to generate a genome assembly or gene sequence. As another example, the accessed sequencing data may include amino acid sequences on which an assembly algorithm was applied to generate a protein sequence. In some embodiments, the system may be configured to access sequencing data generated from a target sequencing technology that the machine learning model trained in process 300 has been optimized to update. As an example, the machine learning model may be trained on sequencing data generated from third generation sequencing, and the system may access sequencing data generated from third generation sequencing.

Next, process 310 proceeds to block 316 where the system generates input to be provided to the machine learning model using the sequencing data and assembly. In some embodiments, the system may be configured to generate inputs for respective locations in the assembly. The system may be configured to generate inputs for a set of locations in the assembly by: (1) aligning sequences from the sequencing data to the set of locations in the assembly; and (2) comparing biological polymers of the aligned sequences to biological polymers indicated at the locations in the assembly to determine values of one or more features. In some embodiments, the system may be configured to align sequences to a set of locations in the assembly by identifying sequences from the sequencing data that indicate biological polymers at the set of locations in the assembly. As an example, the assembly may include locations that are indexed from 1 to 10,000, and the system may determine that nucleotide sequences “TAGGTC”, “TAGTTC”, “TAGGCC”, “TAGGTC” each aligns with locations indexed 5-10 of the assembly. In this example, the system may compare each of the nucleotide sequences to biological polymers indicated at the locations indexed 5-10 in the assembly to determine values of the feature(s). Examples of features, and generation of values of the features are described with reference to FIGS. 4A-C.

In some embodiments, the system may be configured to generate inputs for respective locations in the assembly. The system may be configured to generate an input for a location to provide as input to the machine learning model to obtain output that may be used to identify a biological polymer (e.g., nucleotide, amino acid) that is present at the location in the assembly. In some embodiments, the system may be configured to generate an input for a location in the assembly based on a biological polymer indication at the location, and biological polymer indications at one or more other locations that are in a neighborhood of the location. The input may provide a machine learning model with contextual information around a location in the assembly which the model uses to generate a corresponding output. The system may be configured to generate an input for a location based on biological polymer indications at locations in a neighborhood of the location by determining values of feature(s) at the location and at the other location(s) in the neighborhood of the location. As an example, the system may: (1) select a location; (2) identify a neighborhood of locations centered at the selected location; and (3) generate the input to be values of the feature(s) at each of the selected location and the neighborhood of locations.

In some embodiments, the system may be configured to use a neighborhood of a set size. Example neighborhood sizes are described herein. In some embodiments, the number of locations in a neighborhood used by the system may be a configurable parameter. For example, the system may receive a user input (e.g., in a software application) specifying a neighborhood size to use. In some embodiments, the system may be configured to determine a neighborhood size. As an example, the system may determine a neighborhood size based on a sequencing technology that the sequencing data was generated by and/or an assembly technology that the assembly was generated by.

In some embodiments, the system may be configured to generate the input to be provided to the machine learning model by: (1) selecting locations in the assembly; and (2) generating respective inputs for the selected locations. In some embodiments, the system may be configured to select the locations in the assembly by determining likelihoods that the assembly incorrectly indicates biological polymers at locations in the assembly, and selecting the locations for which to generate an input using the determined likelihoods. As an example, the system may determine whether a likelihood that the assembly incorrectly indicates a biological polymer at a location exceeds a threshold likelihood, and generate an input for the location if the likelihood exceeds the threshold likelihood. In some embodiments, the system may be configured to determine the likelihood that a location incorrectly indicates a biological polymer based on a number of aligned sequences that indicate that the biological polymer is present at the location. The system may determine the likelihood to be a difference between the number of sequences that indicate that the biological polymer is at the location and the total number of sequences. As an example, the assembly may indicate thymine at a location in the assembly based on a consensus from a set of 9 nucleotide sequences where 4 of the nucleotide sequences indicate that thymine is present at the location, 2 of the nucleotide sequences indicate that guanine is present at the location, and 3 of the nucleotide sequences indicate that adenine is present at the location. In this example, the system may determine a likelihood that the assembly incorrectly indicates the biological polymer at the location in the assembly to be the difference between the number of nucleotide sequences that indicate thymine (4) and the total number of nucleotide sequences (9) to obtain a value of 5. The system may determine that 5 is greater than a threshold difference (e.g., 1, 2, 3, 4) and, as a result, generate an input for the location.

In some embodiments, the system may be configured to use a threshold difference of 1, 2, 3, 4, 5, 6, 7, 8, 9, or 10. Some embodiments are not limited to a particular threshold difference. In some embodiments, the threshold difference may be a configurable parameter. The threshold likelihood used by the system may affect the number of locations for which the system generates an input to be provided to the model. As an example, the system may receive a value of the threshold as a user input to a software application. In some embodiments, the system may use a set threshold likelihood. As an example, value of the threshold likelihood may be encoded. In some embodiments, the system may be configured to automatically determine the threshold likelihood. As an example, the system may determine the threshold likelihood based on an assembly technology from which the assembly was generated and/or a sequencing technology from which the sequencing data was generated.

In some embodiments, the system may be configured to generate an input for a location as a 2-D matrix. In some embodiments, each row/column of the matrix may specify values of feature(s) determined at a respective location in the assembly. In some embodiments, the system may be configured to generate the input as an image, where the pixels of the image hold the values of the feature(s). As an example, each row/column of the image may specify values of feature(s) determined at a respective location in the assembly.

Next, process 310 proceeds to block 318 where the system provides the input generated at block 316 to the machine learning model to obtain a corresponding output. In some embodiments, the system may be configured to provide inputs generated for respective locations in the assembly as separate inputs to the machine learning model. As an example, the system may provide a set of feature values determined at a target location and at locations in a neighborhood of the location as input to the machine learning model to obtain a corresponding output for the target location. In some embodiments, the system may be configured to provide inputs generated for multiple locations in parallel (e.g., as described above with reference to FIGS. 2C-D). As an example, the system may: (1) provide a first input generated for a first location to the model; and (2) prior to obtaining a first output corresponding to the first input, provide a second input generated for a second location to the model. In some embodiments, the system may be configured to provide inputs generated for multiple locations sequentially. For example, the system may: (1) provide a first input generated for a first location to the model to obtain a corresponding first output; and (2) after obtaining the first output, provide a second input for a second location to obtain a corresponding second output.

In some embodiments, output corresponding to input provided to the machine learning model may indicate, for each of multiple locations in an assembly, a likelihood that each of one or more biological polymers is present at the location. As an example, the output may indicate, for each of multiple locations in a genome assembly, a likelihood (e.g., a probability) that each of one or more nucleotides (e.g., adenine, guanine, thymine, cytosine) is present at the location. As another example, the output may indicate, for each of multiple locations in a protein sequence, a likelihood that each of one or more amino acids is present in the location. In some embodiments, an output may indicate a likelihood that no biological polymer is present at a location in the assembly. As an example, the system may indicate a likelihood of a “-” character being at the location in the assembly.

In some embodiments, the model may provide outputs corresponding to respective locations in the assembly. The system may provide an input generated for a target location in the assembly, and obtain a corresponding output indicating likelihoods of each of one or more biological polymers being present at the target location. As an example, the system may provide an input generated for a location in a genome assembly and obtain a corresponding output indicating likelihoods that each of a set of 4 possible nucleotides (e.g., adenine, guanine, thymine, cytosine) is present at the location. For example, the likelihood may be probability values of each nucleotide being present at the location.

Next, process 310 proceeds to block 320 where the system identifies biological polymers at locations in the assembly using the output obtained from the model. In some embodiments, the system may be configured to identify the biological polymers at locations in the assembly by identifying, for each of the locations, a biological polymer that is present at the location using an output obtained for the location in response to a corresponding input provided to the model. The output from the model may include multiple sets of output values corresponding to respective locations. Each set of output values may specify likelihoods that each of one or more biological polymers is present at a respective location in the assembly. The system may identify a biological polymer at the respective location to be the biological polymer having the greatest likelihood of being present at the location. As an example, a set of output values for a first location in the assembly may indicate the following set of likelihoods for the location: adenine (A) 0.1, cytosine (C) 0.6, guanine (G) 0.1, thymine (T) 0.15, and blank (-) 0.05. In this example, the system may identify cytosine (C) to be at the location in the assembly. In some embodiments, an output from the model corresponding to an input generated for a location may be a classification specifying a biological polymer at the location. As an example, the output from the model may be a classification of adenine (A), cytosine (C), guanine (G), thymine (T), or blank (-).

Next, process 310 proceeds to block 322, where the system updates the assembly to obtain an updated assembly. The system may be configured to update the assembly based on the identified biological polymers at block 320. In some embodiments, the system may be configured to update the assembly by updating indications of biological polymers at locations in the assembly. In some instances, a biological polymer identified as being present at a location at block 320 may be different than a biological polymer indication in the assembly. In these instances, the system may modify the biological polymer indication at the location in the assembly. As an example, the system may: (1) identify, using the output of the model, that thymine “T” is present at a first location in the assembly which has an indication of adenine “A”; and (2) change the first location in the assembly to indicate from a previous indication of adenine “A” to thymine “T”. In some instances, a biological polymer identified as being present at a location may be the same as the biological polymer indication at the location in the assembly. In these instances, the system may not change the biological polymer indication at the location in the assembly. As an example, the system may: (1) identify, using the output of the model, that thymine “T” is present at a first location in the assembly which has an indication of thymine “T”; and (2) leave the indication at the first location unchanged.

In some embodiments, the system may be configured to update multiple locations in the assembly in parallel. As an example, the system may: (1) begin updating a first location in the assembly; and (2) prior to completing an update at the first location, begin updating a second location in the assembly. In some embodiments, the system may be configured to update locations in the assembly sequentially. As an example, the system may: (1) update a first location in the assembly; and (2) after completing the update at the first location in the assembly, update the second location in the assembly.

In some embodiments, after updating the assembly at block 322 to obtain a first updated assembly, process 310 may return to block 316 as indicated by the dashed line from block 322 to block 316. In some embodiments, the system may be configured to generate input to the machine learning model using the first updated assembly and the sequencing data. As an example, the system may generate input to the model using a set of nucleotide sequences of the sequencing data and the first updated assembly. The system may align the nucleotide sequences to respective locations of the first updated assembly to generate the input to the machine learning model as described above. The system may then perform acts at block 316 to 322 to obtain a second updated assembly. In some embodiments, the assembly system may be configured to perform iterations until a condition is met.

In some embodiments, the system may be configured to perform update iterations until the system determines that a threshold number of iterations have been performed. In some embodiments, the threshold number of iterations may be set by a user input (e.g., a software command, or hard-coded value). In some embodiments, the system may be configured to determine a threshold number of iterations. As an example, the system may determine a threshold number of update iterations based on a type of assembly technique that was used to obtain an initial assembly. In some embodiments, the system may be configured to perform update iterations until the system detects that the assembly has converged. As an example, the assembly system may: (1) determine a number of differences between a current assembly obtained from the latest iteration and a previous assembly; and (2) determine to stop performing update iterations when the number of differences is less than a threshold number or percentage of differences.

In some embodiments, the system may be configured to perform a single update to the assembly, and process 310 may end at block 322 after performing the single update to the assembly. The updated assembly may be output by the system as an output assembly. As an example, the system may output a genome assembly in which errors in the assembly have been corrected such that the output assembly is more accurate than the initial assembly accessed at block 314. As another example, the system may output a protein sequence in which errors have been corrected such that the output protein sequence is more accurate than an initial protein sequence accessed at block 314.

In some embodiments, the system may be configured to perform a first number of update iterations for a first portion of an assembly and a second number of update iterations for a second portion of the assembly. As an example, the system may update locations indexed 1-100 of a genome assembly multiple times (e.g., by performing multiple iterations of acts at blocks 316-322), and update locations indexed 101-200 of the genome assembly once (e.g., by performing the acts at blocks 316-322 once). The system may be configured to determine portions of the assembly to update multiple times based on a number of locations in the portions that may incorrectly indicate biological polymers. As an example, the system may: (1) determine a number of locations in a window of locations (e.g., 25, 50, 75, 100, or 1000 locations) that have a likelihood of having incorrect biological polymer indications that exceeds a threshold likelihood; and (2) determine to perform an update cycle on the window of locations when the number exceeds a threshold number of locations.

FIG. 4A-C show an example of generating input to be provided to a machine learning model, in accordance with some embodiments of the technology described herein.

FIG. 4A shows an array 400 that includes nucleotide sequences 401 (labelled “Pileup” in FIG. 4A), an assembly 402 of biological polymers generated from the nucleotide sequences 401, and labels 404 of biological polymers for respective locations in the assembly. As an example, the data shown in FIG. 4A may be training data obtained from performing process 300 for training a machine learning model where: (1) the sequencing data 401 and assembly 402 are obtained at blocks 302 and 304; (2) and the labels 404 are obtained at block 306. As another example, the sequencing data 401 and assembly 402 may be obtained at blocks 312 and/or 314 of process 310 for generating an assembly using a trained machine learning model.

As shown in the embodiment of FIG. 4A, the sequencing data 401 includes nucleotide sequences generated from sequencing DNA. Each row of the sequencing data 401 is a nucleotide sequence. As shown in the example of FIG. 4A, the nucleotide sequences are represented as sequences of alphanumeric characters where “A” represents adenine, “C” represents cytosine, “G” represented guanine, “T” represents thymine, and “-” represents that no nucleotide is present at the location. Example alphanumeric characters described herein are for illustrative purposes, as some embodiments are not limited to a particular set of alphanumeric characters to represent respective nucleotides or lack thereof.

In the embodiment of FIG. 4A, the assembly 402 is generated from the nucleotide sequences 401. In some embodiments, the assembly 402 may be obtained from applying an assembly algorithm (e.g., OLC assembly) to the sequencing data 401. In the embodiment of FIG. 4A, the assembly 402 is obtained from taking a consensus of the nucleotide sequences. The consensus is determined by a majority vote of the nucleotide sequences for each location in the assembly 402 in which the system identifies the biological polymer indicated at the location by the greatest number of nucleotide sequences. The system may be configured to, for each of multiple nucleotides: (1) determine the number of nucleotide sequences that vote for the nucleotide (e.g., by indicating that the nucleotide is present at the location); and (2) identify the nucleotide having the greatest number of votes to be indicated at the location. As an example, for the location of highlighted column 406: (1) 4 of the sequences indicate adenine, 3 of the sequences indicate cytosine, and 2 of the sequences indicate guanine; and (2) the location in the assembly 402 indicates adenine. As another example, for the first location in the assembly 402, all the nucleotide sequences indicate cytosine and thus the assembly 402 indicates cytosine at the first location.

In the embodiment of FIG. 4A, the labels 404 may indicate desired biological polymers for the locations in the assembly 402. In some embodiments, the system may be configured to determine the labels from a reference genome. For example, the system may obtain the nucleotide sequences from sequencing a DNA sample from an organism, obtain the assembly 402 from application of an assembly algorithm to the nucleotide sequences, and obtain the labels 404 from a known reference genome of the organism (e.g., from the NCBI database). The labels 404 may represent a true or correct biological polymer indication for each location to be used for supervised training and/or for determining an accuracy of a generated assembly.

FIG. 4B shows an array 410 of values determined from the data 400 shown in FIG. 4A. The array 410 illustrates an intermediate step in generation of an input to a machine learning model for the location of column 406 in the assembly 402. The array 410 includes a set of rows labelled “Pileup” representing the nucleotide sequences of FIG. 4A. For each location in the assembly, the system determines a count for each of multiple nucleotides, where the count indicates a number of the nucleotide sequences that indicate that the nucleotide is at the location in the assembly. Each entry in the “Pileup” section of the array 410 holds a count for a nucleotide. As an example, the column 412 in FIG. 4B has a count of 4 for adenine, 3 for cytosine, 2 for guanine, 0 for thymine, and 0 for no nucleotide. As another example, the first column of the array 410 has a count of 0 for adenine, 9 for cytosine, 0 for guanine, 0 for thymine, and 0 for no nucleotide.

The array 410 further includes a set of rows, labelled “Assembly” in FIG. 4B, representing the assembly 402 of FIG. 4B. For each location in the assembly 402, the array 410 includes a column of values determined from the nucleotide indicated at the location. For each location, the system may assign a reference value to each of multiple nucleotides, where the reference value indicates whether the nucleotide is indicated at the location in the assembly. As one example, in the column labelled 412 of FIG. 4B, the assembly section: (1) has a value of 9 for adenine because that is the nucleotide indicated at the corresponding location in the assembly 402; and (2) has a value of 0 for each of the other nucleotides because they are not indicated at the corresponding location in the assembly 402. As another example, the first column of the array 410 the assembly section: (1) has a value of 9 for cytosine because that is the nucleotide indicated at the corresponding location in the assembly 402; and (2) has a value of 0 for each of the other nucleotides because they are not indicated at the corresponding location in the assembly 402. As illustrated in the example of FIG. 4B, in some embodiments, the reference value assigned to a nucleotide at an assembly location when the nucleotide is indicated at the assembly location is equal to the number of aligned nucleotide sequences (e.g., 9 in the example of FIG. 4A).

FIG. 4C shows an array 420 of feature values generated using the values in array 410 of FIG. 4B. In some embodiments, the array 420 may be provided as input to a machine learning model to obtain a corresponding output. In the example of FIG. 4C, the array 420 is the input to be provided to a model for the location in the assembly corresponding to the column 422. The array 420 include values of features determined at a target location corresponding to column 422, and values of features determined for 24 locations in a neighborhood of the target location. The array 420 includes values of features for 12 locations to the left of the target location, and 12 locations to the right of the target location.

In the Pileup section of array 420, each column specifies an error value for each of multiple nucleotides. The error value for a nucleotide in the column indicates a difference between: (1) the number of nucleotide sequences that indicate that the nucleotide is at the location in the assembly 402 corresponding to the column, and (2) the reference value assigned to the nucleotide in the Assembly section of array 420. As an example, for column 422 of FIG. 4C, the values are determined as follows: (1) adenine is 4−9=−5 (2) cytosine is 3−0=3 (3) guanine is 2−0=2; (4) thymine is 0−0=0 (5) blank is 0−0=0. The Assembly section of array 420 may be the same as the Assembly section of array 410 of FIG. 4B.

In some embodiments, the values of the Pileup in array 420 may indicate a likelihood that the assembly 402 incorrectly identifies a nucleotide at a location. The system may select locations for which to generate an input to a machine learning model using the values. As illustrated in FIG. 4C, the non-zero values of the Pileup are highlighted. In some embodiments, the system may be configured determine to generate an input to be provided to the machine learning model for a location when a Pileup value at the location exceeds a threshold value. For example, the system may determine to generate an input for the location in the assembly 402 corresponding to column 422 by determining that a difference of 5 determined for adenine exceeds a threshold difference of 4. Example threshold differences are described herein.

In some embodiments, the array 420 may be provided as input to the machine learning model to update a location in the assembly (e.g., location corresponding to column 422). The system may use a corresponding output obtained from the machine learning model to identify a nucleotide that is present at the location in the assembly, and update the assembly accordingly. In some embodiments, the array 420 may be one of multiple inputs provided to the machine learning model as part of training the model. The system may use a corresponding outputs obtained from the machine learning model and the labels 404 to determine adjustments to one or more parameters of the machine learning model. As an example, the machine learning model may be a neural network, and the system may use the difference between the nucleotides identified from the outputs of the machine learning model and the labels to determine one or more adjustments to weights of the neural network.

Although the example embodiment of FIG. 4A shows data related to nucleic acids, in some embodiments, the data may be related to a protein. For example, the sequences 401 may be amino acids sequences, the assembly 402 may be a protein sequence, and the labels 404 may be reference amino acids for each of the locations in the protein sequence. The system may determine the values shown in FIGS. 4B—C based on the amino acid sequences, the protein sequence, and/or the labels.

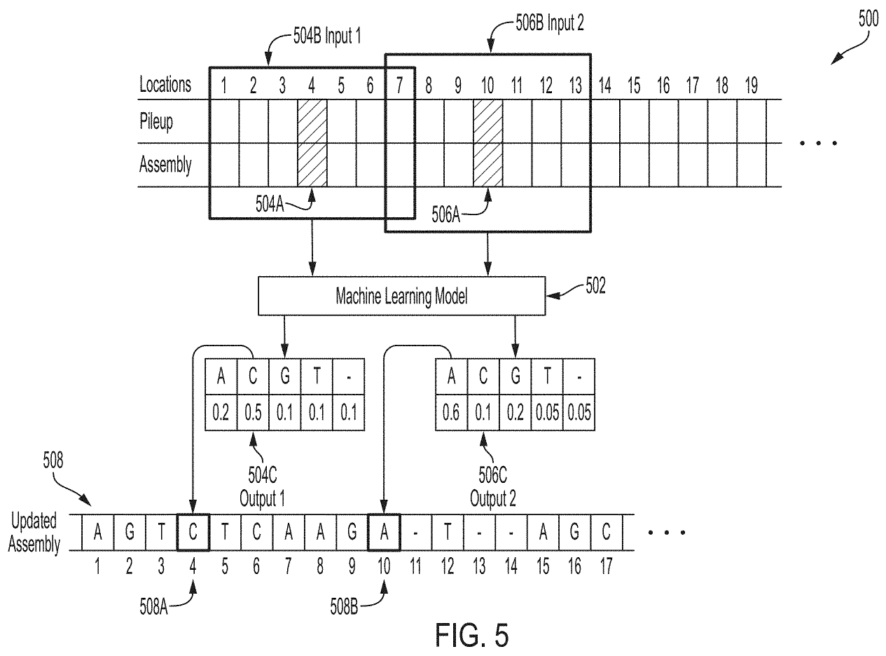

FIG. 5 illustrates a process of updating an assembly, according to some embodiments of the technology described herein. FIG. 5 shows generation of input from assembly data 500 to be provided to a machine learning model 502 to generate an updated assembly 508. The assembly data 500 may be, for example, in the form of data described above with reference to FIG. 4C. The illustrated process of updating may be performed by assembly system 104 described above with reference to FIGS. 1A-C.

As shown in the embodiment of FIG. 5, the system selects the location 504A and 506A in the assembly to be updated. As an example, the system may select the locations 504A, 506A by: (1) determining likelihoods that the assembly incorrectly indicates a biological polymer (e.g., nucleotide, amino acid) at locations in the assembly; and (2) determining that the likelihoods at the location 504A, 506A each exceeds a threshold likelihood to select the locations 504A, 506A. When the system selects the locations 504A, 506A, the system may determine to generate corresponding inputs to be provided to the machine learning model 502.

As shown in the embodiment of FIG. 5, the system generates a first input 504B corresponding to the location 504A and a second input 506B corresponding to the location 506A. The system may generate each of the inputs 504B, 506B as described above with reference to FIGS. 4A-C. For example, the system may generate each of the inputs 504B, 506B by: (1) selecting a neighborhood of locations centered at the location; (2) determining values of one or more features at each of the locations in the neighborhood; and (3) using the values of the feature(s) as the input for the location. In some embodiments, the system may be configured store the values of the feature(s) in a data structure. As an example, the system may store the values in a two dimensional array, or image as illustrated in FIG. 4C.

As shown in the embodiment of FIG. 5, the system provides each of the generated inputs 504B, 506B as input to the machine learning model 502 to obtain corresponding outputs. Output 504C corresponds to the input 504B generated for location 504A, and output 506C corresponds to the input 506B generated from location 506A. In some embodiments, the system may be configured to provide the inputs 504B, 506B to the machine learning model 502 sequentially. As an example, the system may: (1) provide input 504B to the machine learning model 502 to obtain a corresponding output 504C; and (2) after obtaining the output 504C, provide the input 506B to the machine learning model 502 to obtain a corresponding output 506C. In some embodiments, the system may be configured to provide the inputs 504B, 506B to the machine learning model 502 in parallel. As an example, the system may: (1) provide input 504B to the machine learning model 502; and (2) prior to obtaining output 504C corresponding to the input 504B, provide input 506B to the machine learning model 502.

As shown in the embodiment of FIG. 5, each of the outputs 504C, 506C indicates likelihoods that each of one or more nucleotides is present at a location in the assembly. In the embodiment of FIG. 5, the likelihoods are probabilities. As an example, output 504C specifies: (1) for each of four different nucleotides, a probability that the nucleotide is present at the location 504A; and (2) a probability that no nucleotide is present at the location 504A (represented by the “-” character). In output 504C, adenine has a probability of 0.2, cytosine a probability of 0.5, guanine a probability of 0.1, thymine a probability of 0.1, and there is a 0.1 probability that no nucleotide is at the location 504A. As another example, output 506C specifies: (1) for each of four different nucleotides, a probability that the nucleotide is present at the location 506A; and (2) a probability that no nucleotide is present at the location 506A (represented by the “-” character). In this example, adenine has a probability of 0.6, cytosine a probability of 0.1, guanine a probability of 0.2, thymine a probability of 0.05, and there is a 0.05 probability that no nucleotide is at the location 504A.

As shown in the embodiment of FIG. 5, the system uses output obtained from the machine learning model 502 to update locations in the assembly to obtain an updated assembly 508. In some embodiments, the system may be configured to update the assembly by: (1) identifying a nucleotide that is present at locations using the output obtained from the machine learning model; and (2) updating the locations in the assembly to indicate the identified nucleotides to obtain the updated assembly 508. As shown in the example of FIG. 5, the system updates the location 504A in the initial assembly by: (1) determining that cytosine has the highest likelihood of being present at the location using the output 504C; and (2) setting the corresponding location 508A in the updated assembly 508 to indicate cytosine “C” at the location. As another example, the system updates the location 506A in the initial assembly by: (1) determining that adenine has the highest likelihood of being present at the location using the output 506C; and (2) setting the corresponding location 508B in the updated assembly 508 to indicate adenine “A”. In some instances, the system may: (1) determine that the nucleotide identified at a location using output obtained from the machine learning model 502 may already be indicated at the location; and (2) keep the indication at the location unchanged in the updated assembly 508.

Although the updated assembly 508 is shown separate from the initial assembly, in some embodiments, the updated assembly 508 may be an updated version of an initial assembly. For example, the system may store an initial assembly in memory, and update values of the initial assembly in memory to obtain the updated assembly 508. In some embodiments, the system may generate the updated assembly 508 as a separate assembly than an initial assembly. For example, the system may store an initial assembly at a first memory location, and store the updated assembly 508 as a separate assembly at a second memory location.

In some embodiments, the system may be configured to perform updates at locations in an initial assembly sequentially. As an example, the system may: (1) update the location 508A in the updated assembly 508 using the output 504C; and (2) after completing the update at the location 508A, update the location 508B in the updated assembly 508 using the output 506C. In some embodiments, the system may be configured to perform updates at locations in an initial assembly in parallel. As an example, the system may: (1) begin updating the location 508A using the output 504C; and (2) before completing an update at location 508A, begin updating the location 508B using the output 506C.

In some embodiments, the system may be configured to perform a process of generating inputs for respective locations in an assembly, providing the inputs to the machine learning model 502, and updating the locations in the assembly using outputs from the machine learning model in parallel. As an example, the system may: (1) begin generation of an input for location 504A of the initial assembly; and (2) prior to completing an update to the location at location 504A, begin generation of an input for location 506A of the initial assembly. By parallelizing the assembly updates, the system makes the process of generating an assembly more efficient (e.g., by requiring less time). The system may parallelize processes by using multiple processors and/or using multiple application threads.

Although the embodiment of FIG. 5 illustrates updating a portion of a genome assembly, some embodiments may implement the illustrated process to update a protein sequence or portion thereof. For example, the initial assembly may be a protein sequence. The system may then generate inputs for locations in the protein sequence to provide to the machine learning model 502. The system may obtain output indicating likelihoods (e.g., probabilities) that each of multiple amino acids is present at the location. The system may then update the initial protein sequence to obtain an updated protein sequence.

FIG. 6 illustrates an example of a convolutional neural network model 600 for generating an assembly, according to some embodiments of the technology described herein. In some embodiments, the convolutional neural network model 600 may be trained by performing process 300 described above with reference to FIG. 3A. In some embodiments, the trained convolutional neural network model 600 obtained from process 300 may be used to perform process 310 to generate an assembly as described above with reference to FIG. 3B.

In some embodiments, the model 600 is configured to receive input generated from sequencing data, and an assembly generated from the sequencing data. As an example, the model 600 may be a machine learning model used by the assembly system 104 described above with reference to FIGS. 1A-C. The sequencing data may include biological polymer sequences (e.g., nucleotide sequences or amino acid sequences). In some embodiments, the system may be configured to determine values of one or more features, and provide the determined values as input to the model 600. As an example, the system may determine values of features at a neighborhood of locations in an assembly and provide the determined values at the neighborhood of locations as input to the model 600. Example inputs and techniques for generating inputs are described herein.

In the example embodiment of FIG. 6, the model 600 includes a first convolutional layer 602 which receives input provided to the model 600. In the first layer 602, the system convolves input provided to the model 600 with 64 3×5 filters represented as a 3×5×64 matrix. For example, the system may convolve a 10×25 input matrix (e.g., as illustrated in FIG. 4C) with each channel of the 3×5×64 matrix to obtain an output. The first layer 602 includes a ReLu function as an activation function that the system applies to the output from the convolution. In some embodiments, the first layer 602 may also include a pooling layer to reduce the size of the output of the convolution.

In the example embodiment of FIG. 6, the model includes a second convolutional layer 604 that receives the output of the first layer 602. In the second layer 604, the system convolves the input with a set of 128 3×5 filters represented as a 3×5×128 matrix. The system may convolve the output from the first convolutional layer 602 with the 3×5×128 filter set. The second convolutional layer 604 includes a ReLU function as an activation function that the system applies to the output from the convolution. In some embodiments, the second layer 604 may also include a pooling layer to reduce the size of the output of the convolution. The output of the second convolutional layer 604 is then passed to a third convolutional layer 606. In third layer 606, the system convolves the input with a set of 256 3×5 filters represented as a 3×5×256 matrix. The system then applies a ReLu activation function to the output from the convolution. In some embodiments, the third layer 606 may also include a pooling layer to reduce the size of the output of the convolution.

In the example embodiment of FIG. 6, the model 600 includes a dense layer 608 having 5 fully connected layers, each of which receives 256 input values. The system may condense an output obtained from the third convolutional layer 606 to provide as input to the dense layer 608. The dense layer 608 may output multiple values, where each value indicates a likelihood of a respective biological polymer (e.g., nucleotide or amino acid) being present at a location for which an input was provided to the model 600. As an example, the dense layer may output five values, where each value indicates a likelihood of a nucleotide (e.g., adenine, cytosine, guanine, thymine, and/or no nucleotide) being present at the location. The system may apply a softmax function to the output of the dense layer 608 to obtain a set of probability values that sum to 1. As shown in the example embodiment of FIG. 6, the system applies a softmax function to the output of the dense layer 608 to obtain an output 610 of 5 probabilities indicating probabilities that respective nucleotides are present at a location in an assembly. The output 610 may be used to update an assembly (e.g., as described above with reference to FIG. 5).

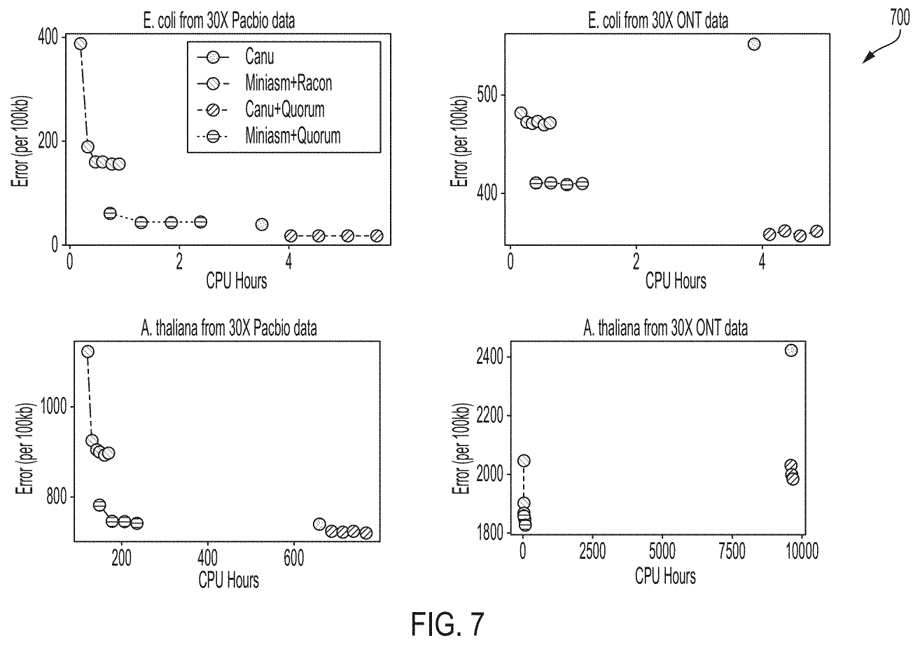

FIG. 7 illustrates performance results of techniques in accordance with some embodiments of the technology described herein. Each of the plots shows improvements in accuracy provided by the techniques relative to conventional techniques. In FIG. 7, Canu and Miniasm are two conventional assembly techniques. Miniasm+Racon represent Miniasm with application of Racon error correction. Canu+Quorum is an implementation of techniques described herein to correct an assembly generated from Canu. Miniasm+Quorum is an implementation of techniques described herein to correct an assembly generated from Miniasm.

As illustrated in FIG. 7, Miniasm+Quorum has significantly fewer error rates than Miniasm+Racon for each of the samples of data. As an example, for E. coli from 30× Pacbio data, each iteration of Miniasm+Quorum (represented by connected points) has error rates of less than 100 errors/100 kilo-bases, while Miniasm+Racon has a minimum error rate of approximately 200 errors/100 kilo-bases. As another example, for E. coli from 30×ONT data, each iteration of Miniasm+Quorum has error rates of approximately 400 errors/100 kilo-bases, whereas Miniasm+Racon has error rates of approximately 500 errors/100-kilo-bases.

As illustrated in FIG. 7, Canu+Quorum provides improved accuracy over results from Canu alone. Although Canu includes incorporates conventional error correction techniques, techniques described herein provide improved accuracy of assembly generation. As an example, for E. coli from 30×ONT data, Canu has an error rate of greater than 500 errors/100 kilo-bases while each iteration of Canu+Quorum has an error rate of less than 350 errors/100 kilo-bases.

As illustrated in FIG. 7, techniques described herein may provide improved accuracy of assemblies without adding substantially large amounts of computation time to perform error correction. As an example, Miniasm+Quorum achieves better accuracy than Miniasm+Racon in substantially the same number of CPU hours. As another example, Canu+Quorum achieves better accuracy than Canu alone without substantially increasing the number of CPU hours to correct the assembly.

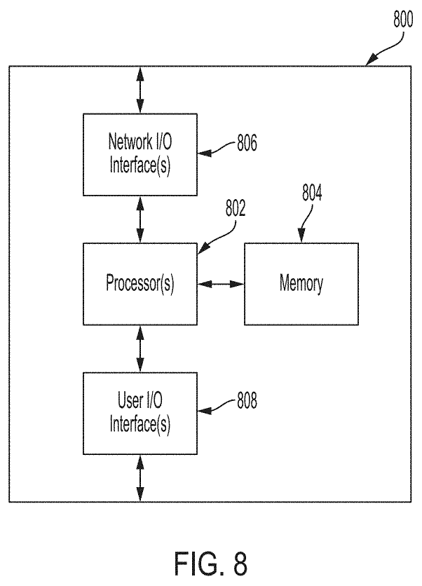

In some embodiments, systems and techniques described herein may be implemented using one or more computing devices. Embodiments are not, however, limited to operating with any particular type of computing device. By way of further illustration, FIG. 8 is a block diagram of an illustrative computing device 800. Computing device 800 may include one or more processors 802 and one or more tangible, non-transitory computer-readable storage media (e.g., memory 804). Memory 804 may store, in a tangible non-transitory computer-recordable medium, computer program instructions that, when executed, implement any of the above-described functionality. Processor(s) 802 may be coupled to memory 804 and may execute such computer program instructions to cause the functionality to be realized and performed.

Computing device 800 may also include a network input/output (I/O) interface 806 via which the computing device may communicate with other computing devices (e.g., over a network), and may also include one or more user I/O interfaces 808, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. As an example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discus sed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. As an example, aspects described in one embodiment may be combined in any manner with aspects described in other embodiments.

The terms “approximately”, “substantially,” and “about” may be used to mean within ±20% of a target value in some embodiments, within ±10% of a target value in some embodiments, within ±5% of a target value in some embodiments, and within ±2% of a target value in some embodiments. The terms “approximately” and “about” may include the target value.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

What is claimed is:

1. A method of generating a biological polymer assembly of a macromolecule, the method comprising:
using at least one computer hardware processor to perform:
accessing a plurality of biological polymer sequences and an assembly indicating biological polymers present at respective assembly locations;
generating, using the plurality of biological polymer sequences and the assembly, a first input to be provided to a trained deep learning model;
providing the first input to the trained deep learning model to obtain a corresponding first output indicating, for each of a first plurality of assembly locations, one or more likelihoods that each of one or more respective biological polymers is present at the location;
identifying biological polymers at the first plurality of assembly locations using the first output of the trained deep learning model; and
updating the assembly to indicate the identified biological polymers at the first plurality of assembly locations to obtain an updated assembly.

2. The method of claim 1, wherein the macromolecule comprises a protein, the plurality of biological polymer sequences comprises a plurality of amino acid sequences, and the assembly indicates amino acids at respective assembly locations.

3. The method of claim 1, wherein the macromolecule comprises a nucleic acid, the plurality of biological polymer sequences comprises a plurality of nucleotide sequences, and the assembly indicates nucleotides at respective assembly locations.

4. The method of claim 3, wherein:
the assembly indicates a first nucleotide at a first one of the first plurality of assembly locations;
identifying the biological polymers at the first plurality of assembly locations comprises identifying a second nucleotide at the first assembly location; and
updating the assembly comprises updating the assembly to indicate the second nucleotide at the first assembly location.

5. The method of claim 3, further comprising, after updating the assembly to obtain the updated assembly:
aligning the plurality of nucleotide sequences to the updated assembly;
generating, using the plurality of nucleotide sequences and the updated assembly, a second input to be provided to the trained deep learning model;
providing the second input to the trained deep learning model to obtain a corresponding second output indicating, for each of a second plurality of assembly locations, one or more likelihoods that each of one or more respective nucleotides is present at the location;
identifying nucleotides at the second plurality of assembly locations based on the second output of the trained deep learning model; and
updating the updated assembly to indicate the identified nucleotides at the second plurality of assembly locations to obtain a second updated assembly.

6. The method of claim 3, further comprising aligning the plurality of nucleotide sequences to the assembly.

7. The method of claim 3, wherein generating the first input to the trained deep learning model comprises:
selecting the first plurality of assembly locations; and
generating the first input based on the selected first plurality of assembly locations.

8. The method of claim 7, wherein selecting the first plurality locations in the assembly comprises:
determining likelihoods that the assembly incorrectly indicates nucleotides at the first plurality of assembly locations; and
selecting the first plurality of assembly locations using the determined likelihoods.

9. The method of claim 3, wherein generating the first input to be provided to the trained deep learning model comprises comparing respective ones of the plurality of nucleotide sequences to the assembly.

10. The method of claim 3, wherein generating the first input to be provided to the trained deep learning model to identify a nucleotide at a first one of the first plurality of assembly locations comprises:
for each of multiple nucleotides at each of one or more assembly locations in a neighborhood of the first assembly location:
determining a count indicating a number of the plurality of nucleotide sequences that indicate that the nucleotide is at the location;
determining a reference value based on whether the assembly indicates the nucleotide at the location;
determining an error value indicating a difference between the count and the reference value; and
including the reference value and the error value in the first input.

11. The method of claim 10, wherein determining the reference value based on whether the assembly indicates the nucleotide at the location comprises:
determining the reference value to be a first value when the assembly indicates the nucleotide at the location; and
determining the reference value to be a second value when the assembly does not indicate the nucleotide at the location.

12. The method of claim 10, wherein generating the first input to be provided to the trained deep learning model comprises arranging values into a data structure having columns, wherein:
a first column holds reference values and error values determined for the multiple nucleotides at the first assembly location; and
a second column holds reference values and error values determined for the multiple nucleotides at a second one of the one or more assembly locations in the neighborhood of the first assembly location.

13. The method of claim 3, further comprising generating the assembly from the plurality of nucleotide sequences, the generating comprising determining a consensus sequence from the plurality of nucleotide sequences to be the assembly.

14. The method of claim 1, further comprising:
accessing training data including biological polymer sequences obtained from sequencing a reference macromolecule and a predetermined assembly of the reference macromolecule; and
training a deep learning model using the training data to obtain the trained deep learning model.

15. The method of claim 1, wherein the deep learning model comprises a convolutional neural network (CNN).

16. A system for generating a biological polymer assembly of a macromolecule, the system comprising:
at least one computer hardware processor; and
at least one non-transitory computer-readable storage medium storing instructions that, when executed by the at least one computer hardware processor, cause the at least one computer hardware processor to perform:
accessing a plurality of biological polymer sequences and an assembly indicating biological polymers present at respective assembly locations;
generating, using the plurality of biological polymer sequences and the assembly, a first input to be provided to a trained deep learning model;
providing the first input to the trained deep learning model to obtain a corresponding first output indicating, for each of a first plurality of assembly locations, one or more likelihoods that each of one or more respective biological polymers is present at the location;
identifying biological polymers at the first plurality of assembly locations using the first output of the trained deep learning model; and
updating the assembly to indicate the identified biological polymers at the first plurality of assembly locations to obtain an updated assembly.

17. The system of claim 16, wherein the macromolecule comprises a nucleic acid, the plurality of biological polymer sequences comprises a plurality of nucleotide sequences, and the assembly indicates nucleotides at respective assembly locations.

18. The system of claim 16, wherein the instructions further cause the at least one computer hardware processor to perform, after updating the assembly to obtain the updated assembly:
aligning the plurality of nucleotide sequences to the updated assembly;
generating, using the plurality of nucleotide sequences and the updated assembly, a second input to be provided to the trained deep learning model;
providing the second input to the trained deep learning model to obtain a corresponding second output indicating, for each of a second plurality of assembly locations, one or more likelihoods that each of one or more respective nucleotides is present at the location;
identifying nucleotides at the second plurality of assembly locations based on the second output of the trained deep learning model; and
updating the updated assembly to indicate the identified nucleotides at the second plurality of assembly locations to obtain a second updated assembly.

19. The system of claim 16, wherein generating the first input to be provided to the trained deep learning model to identify a nucleotide at a first one of the first plurality of assembly locations comprises:
for each of multiple nucleotides at each of one or more assembly locations in a neighborhood of the first assembly location:
determining a count indicating a number of the plurality of nucleotide sequences that indicate that the nucleotide is at the location;
determining a reference value based on whether the assembly indicates the nucleotide at the location;
determining an error value indicating a difference between the count and the reference value; and
including the reference value and the error value in the first input.

20. At least one non-transitory computer-readable storage medium storing instructions that, when executed by at least one computer hardware processor, cause the at least one computer hardware processor to perform a method of generating a biological polymer assembly of a macromolecule, the method comprising:
accessing a plurality of biological polymer sequences and an assembly indicating biological polymers present at respective assembly locations;
generating, using the plurality of biological polymer sequences and the assembly, a first input to be provided to a trained deep learning model;
providing the first input to the trained deep learning model to obtain a corresponding first output indicating, for each of a first plurality of assembly locations, one or more likelihoods that each of one or more respective biological polymers is present at the location;
identifying biological polymers at the first plurality of assembly locations using the first output of the trained deep learning model; and
updating the assembly to indicate the identified biological polymers at the first plurality of assembly locations to obtain an updated assembly.
